SEQUENCE LISTING (13 sequences)

SEQ ID NO: 1 - DNA, 20 bp, synthetic construct
  misc_feature 1..20: note = Synthetic Polynucleotide
  misc_feature 20: note = Spacer 18
  source 1..20: mol_type = other DNA
tttttttttttttttttttt

SEQ ID NO: 2 - DNA, 21 bp, synthetic construct
  misc_feature 1..21: note = Synthetic Polynucleotide
  misc_feature 21: note = hexaethyleneglycol
  source 1..21: mol_type = other DNA
ggtggtggtggtggtggtggt

SEQ ID NO: 3 - DNA, 20 bp, synthetic construct
  misc_feature 1..20: note = Synthetic Polynucleotide
  misc_feature 20: note = hexaethyleneglycol
  source 1..20: mol_type = other DNA
aaaaaaaaaaaaaaaaaaaa

SEQ ID NO: 4 - DNA, 21 bp, synthetic construct
  misc_feature 1..21: note = Synthetic Polynucleotide
  misc_feature 21: note = Spacer 18
  source 1..21: mol_type = other DNA
aataataataataataataat

SEQ ID NO: 5 - DNA, 20 bp, synthetic construct
  misc_feature 1..20: note = Synthetic Polynucleotide
  source 1..20: mol_type = other DNA
tttttttttttttttttttt

SEQ ID NO: 6 - DNA, 21 bp, synthetic construct
  misc_feature 1..21: note = Synthetic Polynucleotide
  source 1..21: mol_type = other DNA
ggtggtggtggtggtggtggt

SEQ ID NO: 7 - DNA, 20 bp, synthetic construct
  misc_feature 1..20: note = Synthetic Polynucleotide
  source 1..20: mol_type = other DNA
aaaaaaaaaaaaaaaaaaaa

SEQ ID NO: 8 - DNA, 21 bp, synthetic construct
  misc_feature 1..21: note = Synthetic Polynucleotide
  source 1..21: mol_type = other DNA
aataataataataataataat

SEQ ID NO: 9 - DNA, 20 bp, Mus musculus
  source 1..20: mol_type = genomic DNA
tccatgacgttcctgacgtt

SEQ ID NO: 10 - DNA, 20 bp, synthetic construct
  misc_feature 1..20: note = Synthetic Polynucleotide
  misc_feature 20: note = (Sp18) C3 Thiol
  source 1..20: mol_type = other DNA
tccatgacgttcctgacgtt

SEQ ID NO: 11 - DNA, 20 bp, synthetic construct
  misc_feature 1..20: note = Synthetic Polynucleotide
  misc_feature 20: note = (Sp18)-Cy5- C3Thiol
  source 1..20: mol_type = other DNA
tccatgacgttcctgacgtt

SEQ ID NO: 12 - DNA, 20 bp, synthetic construct
  misc_feature 1..20: note = Synthetic Polynucleotide
  misc_feature 20: note = (Sp18) C3 Thiol
  source 1..20: mol_type = other DNA
tttttttttttttttttttt

SEQ ID NO: 13 - DNA, 24 bp, Homo sapiens
  source 1..24: mol_type = genomic DNA
tcgtcgttttgtcgttttgtcgtt

(19) United States
(12) Patent Application Publication
(10) Pub. No.: US 2024/0398918 A1
(43) Pub. Date: Dec. 5, 2024

(54) DESIGN OF IMMUNOSTIMULATORY PROTEIN-CORE SPHERICAL NUCLEIC ACIDS

(71) Applicant: NORTHWESTERN UNIVERSITY, Evanston, IL (US)

(72) Inventors: Chad A. Mirkin, Wilmette, IL (US); Shuya Wang, Evanston, IL (US); Kacper Skakuj, Durham, NC (US); Xiaoyi S. Hu, Acton, MA (US)

(21) Appl. No.: 18/784,244

(22) Filed: Jul. 25, 2024

Related U.S. Application Data
(63) Continuation of application No. 16/886,712, filed on May 28, 2020, now abandoned.
(60) Provisional application No. 62/964,417, filed on Jan. 22, 2020.

(51) Int. Cl. A61K 39/00; A61K 39/39; A61P 35/00

(52) U.S. Cl. CPC A61K 39/0011; A61K 39/39; A61P 35/00; A61K 2039/55561; A61K 2039/804

(57) ABSTRACT

The disclosure is generally directed to immunostimulatory protein-core spherical nucleic acids (SNAs) comprising a protein core and a ratio of immunostimulatory and non-immunostimulatory strands, methods of making the immunostimulatory protein-core SNAs as well as their use.

### CROSS-REFERENCE TO RELATED APPLICATIONS

This application is a Continuation of U.S. application Ser. No. 16/886,712, filed May 28, 2020, which claims the priority benefit under 35 U.S.C. § 119(e) of U.S. Provisional Patent Application No. 62/964,417, filed Jan. 22, 2020, which are incorporated herein by reference in their entirety.

### STATEMENT OF GOVERNMENT INTEREST

This invention was made with government support under U54CA199091-01 awarded by the National Institutes of Health and N00014-15-1-0043 awarded by the Office of Naval Research. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2020-010A_Seqlisting.xml”, which was created on Jul. 17, 2024 and is 19,373 bytes in size. The subject matter of the Sequence Listing is incorporated herein in its entirety by reference.

### BACKGROUND

Fighting cancer through immunotherapy, by stimulating and training a patient's own immune system to fight cancer cells, is a promising therapeutic approach. Compared to other treatment approaches to fighting cancer (i.e., surgery, chemotherapy, and irradiation), immunotherapy holds potential advantages such as being a systemic, targeted, versatile modality potentially having a memory system. A major remaining challenge for this therapeutic modality is the delivery of the antigen (targeting moiety) and adjuvant (immunostimulatory moiety) in a safe and efficacious manner.

### SUMMARY

Immunotherapies against cancer are an exciting new method for the treatment of cancer. This novel therapeutic modality trains the patient's own immune system to attack the tumor. This disclosure describes a spherical nucleic acid (SNA) construct with an antigenic protein as the core (which targets the immune response against cancer cells) with oligonucleotides attached to its surface. The oligonucleotides attached to the surface of the protein can be both immune stimulating (adjuvant sequence such as CpG, which activate the immune system) or non-immune stimulating (non-specific sequence such as T20).

This protein-core SNA construct elicits a stronger immune response compared to a simple mixture of the individual components (protein and DNA) at equivalent concentrations, indicating the immunostimulatory protein-core SNA architecture is a more efficacious way of activating the immune system. Increased density of oligonucleotides results in stronger immune stimulation, even if the amount of active components (antigen protein and adjuvant oligonucleotides) remain constant. Furthermore, initial observations suggest that similar immune stimulation can be achieved with lower amounts of CpG by adjusting the ratio of adjuvant and non-specific strands. This could potentially be useful if one wants to elicit a robust T-cell or B-cell response while using a lower amount of adjuvant oligonucleotides. Furthermore, the type of response achieved (CD4+ versus CD8+ T-cell activation) may depend on the surface ratio of adjuvant to non-specific oligonucleotides, this is important because the distribution of cell types involved in an immune response has an effect on downstream disease outcomes (e.g., tumor regression, etc.).

Applications for the technology disclosed herein include, but are not limited to, therapeutic or prophylactic protein vaccines for cancer and other diseases, including: HPV, prostate cancer, lung cancer and melanoma.

Advantages of the technology disclosed herein include, but are not limited to:
Protein-core SNAs of the disclosure are more potent immune stimulators than their individual components mixed together at the same concentrations. Protein core SNAs are more potent and deliver multiple antigen epitopes which can increase immune system efficacy against cancer targets.
Increased amount of surface-conjugated oligonucleotide results in stronger immune stimulation
Similar immune stimulation is generated with fewer immunostimulatory strands by using non-immunostimulatory filler strands
Protein-core SNAs of the disclosure generate equivalent or higher immune responses while using the same amounts of adjuvant oligonucleotides, this can be advantageous if one wants to elicit a robust T-cell or B-cell response while minimizing the amount of immune stimulating DNA injected (which has been associated with adverse immune responses)
The type of immune response achieved (CD4+ versus CD8+ T-cell activation) may depend on the surface ratio of adjuvant to non-specific oligonucleotides, this is important because the distribution of cell types involved in an immune response has an effect on downstream disease outcomes (i.e.: tumor regression, etc.). This technology may allow one to tune the type of response by changing the SNA structure.

In various aspects and embodiments of the disclosure, the methods provided herein allow for the design of SNAs for controlling tumor growth and initiation, maximizing the potency of immunotherapeutic drugs, and specifically targeting virus-derived cancer type (CD4+ T cells and B cells associated) or non-virus-derived cancer types (mainly CD8+ T cells associated).

In some aspects, the disclosure provides an immunostimulatory protein-core spherical nucleic acid (IP-SNA) comprising: a protein core; and a shell of oligonucleotides attached to the protein core, wherein the shell of oligonucleotides comprises a ratio of immunostimulatory oligonucleotides to non-immunostimulatory oligonucleotides that is between 100:1 and 1:100. In some embodiments, the protein comprises an antigen that is a tumor associated antigen, a tumor specific antigen, a viral antigen, a neoantigen, or a combination thereof. In some embodiments, the antigen is OVA1, MSLN, p53, Ras, a melanoma related antigen, a HPV related antigen, a prostate cancer related antigen, an ovarian cancer related antigen, a breast cancer related antigen, a hepatocellular carcinoma related antigen, a bowel cancer related antigen, a lung cancer related antigen, an osteocarcinoma related antigen, human papillomavirus (HPV) E6/E7 nuclear protein, or a combination thereof. In some embodiments, the ratio of immunostimulatory oligonucleotides to non-immunostimulatory oligonucleotides is about 2:5. In some embodiments, the ratio of immunostimulatory oligonucleotides to non-immunostimulatory oligonucleotides is about 1:1. In some embodiments, the ratio of immunostimulatory oligonucleotides to non-immunostimulatory oligonucleotides is about 2:4. In some embodiments, at least one oligonucleotide of the shell of oligonucleotides is attached to the protein core through a linker. In some embodiments, each oligonucleotide of the shell of oligonucleotides is attached to the protein core through a linker. In further embodiments, the linker is a cleavable linker, a non-cleavable linker, a traceless linker, or a combination thereof. In some embodiments, the linker is a carbamate alkylene dithiolate linker. In some embodiments, at least one oligonucleotide of the shell of oligonucleotides comprises protein-core-NH—C(O)—O—C₂₋₅alkylene-S—S—C₂₋₇alkylene-Oligonucleotide, or protein-core-NH—C(O)—O—CH₂—Ar—S—S—C₂₋₇alkylene-Oligonucleotide, and Ar comprises a meta- or para-substituted phenyl. In some embodiments, at least one oligonucleotide of the shell of oligonucleotides comprises protein-core-NH—C(O)—O—C(ZA)(ZB)C₁₋₄alkylene-C(XA)(XB)—S—S—C(YA)(YB)C₁₋₆alkylene-Oligonucleotide, and ZA, ZB, XA, XB, YA, and YB are each independently H, Me, Et, or Pr. In some embodiments, at least one oligonucleotide of the shell of oligonucleotides comprises protein-core-NH—C(O)—O—C(XA)(XB)—Ar—S—S—C(YA)(YB)C₂₋₆alkylene-Oligonucleotide, and XA, XB, YA, and YB are each independently H, Me, Et, or Pr. In some embodiments, the linker is an amide alkylene dithiolate linker. In some embodiments, at least one oligonucleotide of the shell of oligonucleotides comprises protein-core-NH—C(O)— C₂₋₅alkylene-S—S—C₂₋₇alkylene-Oligonucleotide. In some embodiments, at least one oligonucleotide of the shell of oligonucleotides comprises protein-core-NH—C(O)— C₁₋₄alkylene-C(XA)(XB)—S—S—C(YA)(YB)C₁₋₆alkylene-Oligonucleotide, and XA, XB, YA and YB are each independently H, Me, Et, or Pr. In some embodiments, the linker is an amide alkylene thioether linker. In some embodiments, at least one oligonucleotide of the shell of oligonucleotides comprises protein-core-NH—C(O)— C₂₋₄alkylene-N-succinimidyl-S—C₂₋₆alkylene-Oligonucleotide. In some embodiments, each of the immunostimulatory oligonucleotides is a toll-like receptor (TLR) agonist. In some embodiments, at least one of the immunostimulatory oligonucleotides is a toll-like receptor (TLR) agonist. In some embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, each of the immunostimulatory oligonucleotides comprises a CpG nucleotide sequence. In some embodiments, at least one of the immunostimulatory oligonucleotides comprises a CpG nucleotide sequence. In some embodiments, each of the non-immunostimulatory oligonucleotides comprises a sequence that is 5′-TTTTTTTTTTTTTTTTTTTT-Spacer 18-3′ (“720”; SEQ ID NO: 1), 5′-(GGT)₇-hexaethyleneglycol-3′ (SEQ ID NO: 2), 5′-AAAAAAAAAAAAAAAAAAAA-hexaethyleneglycol-3′ (“A20”; SEQ ID NO: 3), or 5′-(AAT)₇-Spacer 18-3′ (SEQ ID NO: 4). In some embodiments, at least one of the non-immunostimulatory oligonucleotides comprises a sequence that is 5′-TTTTTTTTTTTTTTTTTTTT-Spacer 18-3′ (“T20”; SEQ ID NO: 1), 5′-(GGT)₇-hexaethyleneglycol-3′ (SEQ ID NO: 2), 5′-AAAAAAAAAAAAAAAAAAAA-hexaethyleneglycol-3′ (“A20”; SEQ ID NO: 3), or 5′-(AAT)₇-Spacer 18-3′ (SEQ ID NO: 4). In some embodiments, each oligonucleotide in the shell of oligonucleotides is single-stranded DNA or double-stranded DNA. In some embodiments, at least one oligonucleotide in the shell of oligonucleotides is single-stranded DNA or double-stranded DNA. In some embodiments, each oligonucleotide in the shell of oligonucleotides is single-stranded RNA or double-stranded RNA. In some embodiments, at least one oligonucleotide in the shell of oligonucleotides is single-stranded RNA or double-stranded RNA. In some embodiments, oligonucleotides in the shell of oligonucleotides are single-stranded DNA, double-stranded DNA, single-stranded RNA, double-stranded RNA, or a combination thereof. In some embodiments, the shell of oligonucleotides comprises about 2 to about 20 oligonucleotides. In some embodiments, the shell of oligonucleotides comprises 7 oligonucleotides. In some embodiments, the shell of oligonucleotides consists of 7 oligonucleotides.

In some aspects, the disclosure provides a composition comprising a plurality of the immunostimulatory protein-core spherical nucleic acids (IP-SNAs) of the disclosure. In some embodiments, at least two of the IP-SNAs comprise a different protein core.

In some aspects, the disclosure provides a pharmaceutical formulation comprising an immunostimulatory protein-core SNA (IP-SNA) or a composition of the disclosure, and a pharmaceutically acceptable carrier or diluent.

In some aspects, the disclosure provides an antigenic composition comprising an immunostimulatory protein-core SNA (IP-SNA) of the disclosure, a composition of the disclosure in a pharmaceutically acceptable carrier, diluent, stabilizer, preservative, or adjuvant, or a pharmaceutical formulation of the disclosure, wherein the antigenic composition is capable of generating an immune response including antibody generation or a protective immune response in a mammalian subject. In some embodiments, the antibody response is a neutralizing antibody response or a protective antibody response.

In some aspects, the disclosure provides a method of producing an immune response to a disease in a subject, comprising administering to the subject an effective amount of an antigenic composition of the disclosure, thereby producing an immune response to the disease in the subject. In some embodiments, the disease is an infection or an immunodeficiency disease. In some embodiments, the disease is cancer. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

In some aspects, the disclosure provides a method of treating a disease in a subject in need thereof, comprising administering to the subject an effective amount of an immunostimulatory protein-core SNA (IP-SNA), a composition, a pharmaceutical formulation, or an antigenic composition of the disclosure, thereby treating the disease in the subject. In some embodiments, the disease is an infection or an immunodeficiency disease. In some embodiments, the infection is Anthrax, Chickenpox, Common cold, Diphtheria, E. coli infection, Giardiasis, HIV/AIDS, Infectious, mononucleosis, Influenza (flu), Lyme disease, Malaria, Measles, Meningitis, Mumps, Poliomyelitis (polio), Pneumonia, Rocky mountain spotted fever, Rubella (German measles), Salmonella infections, Severe acute respiratory syndrome (SARS), Sexually transmitted diseases, Shingles (herpes zoster), Tetanus, Toxic shock syndrome, Tuberculosis, Viral hepatitis, West Nile virus, Whooping cough (pertussis), or a combination thereof. In some embodiments, the immunodeficiency disease is ataxia-telangiectasia, chediak-Higashi syndrome, combined immunodeficiency disease, complement deficiencies, DiGeorge syndrome, hypogammaglobulinemia, Job syndrome, leukocyte adhesion defects, panhypogamma globulinemia, Bruton's disease, congenital agammaglobulinemia, selective deficiency of IgA, Wiskott-Aldrich syndrome, or a combination thereof. In some embodiments, the disease is cancer. In some embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the administering is subcutaneous, intravenous, intraperitoneal, intranasal, or intramuscular.

In some aspects, the disclosure provides a method of stimulating a CD8 T-Cell response in a subject having cancer, comprising administering to the subject an effective amount of an immunostimulatory protein-core SNA (IP-SNA), a composition, a pharmaceutical formulation, or an antigenic composition of the disclosure, wherein the ratio of immunostimulatory oligonucleotides to non-immunostimulatory oligonucleotides is less than or equal to 1, thereby stimulating the CD8 T-cell response in the subject. In some embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the ratio of immunostimulatory oligonucleotides to non-immunostimulatory oligonucleotides is about 1:1. In some embodiments, the ratio of immunostimulatory oligonucleotides to non-immunostimulatory oligonucleotides is about 2:5.

In some aspects, the disclosure provides a method of stimulating a CD4 T-Cell response in a subject having a viral infection, comprising administering to the subject an effective amount of an immunostimulatory protein-core SNA (IP-SNA), a composition, a pharmaceutical formulation, or an antigenic composition of the disclosure, wherein the ratio of immunostimulatory oligonucleotides to non-immunostimulatory oligonucleotides is greater than 1, thereby stimulating the CD4 T-cell response in the subject. In some embodiments, the ratio of immunostimulatory oligonucleotides to non-immunostimulatory oligonucleotides is about 1:0. In some embodiments, the ratio of immunostimulatory oligonucleotides to non-immunostimulatory oligonucleotides is about 2:1. In some embodiments, the viral infection is influenza, HIV, pneumonia virus, human papilloma virus (HPV), or a virus that causes cancer. In some embodiments, the virus that causes cancer is human papilloma virus, Epstein-Barr virus, hepatitis B virus, human herpes virus-8, hepatitis C virus, or a combination thereof.

### BRIEF DESCRIPTION OF THE FIGURES

FIG. 1 depicts exemplary protein-core SNA structures of the disclosure and shows structure-function relationships of the protein-core SNAs.

FIG. 2 is a schematic depicting the activation of T cells using immunostimulatory protein-core SNAs (IP-SNAs). Cancer immunotherapy requires the activation of T-cells targeted against cancer cells with an antigen and adjuvant.

FIG. 3 is a schematic showing the use of a protein-core SNA as described herein as a cancer immunotherapeutic by activating T cells to target cancer cells.

FIG. 4 is a schematic depicting an exemplary synthesis of a protein-core SNA. The figure shows that protein-core SNA structure may be realized through multiple rounds of a two-step synthesis.

FIG. 5 shows results of experiments in which protein-core SNAs of the disclosure were synthesized with two linkers and their chemical responsiveness to reduction was tested.

FIGS. 6A and 6B show the development of protein-core-SNA synthesis (6A) and purification (6B) methods.

FIG. 7 provides results of experiments showing that the structure of protein-core SNAs can be used to increase their immunostimulatory potency of T cell proliferation in vitro.

FIG. 8 shows results from an in vitro T cell proliferation study suggesting improved immunostimulation with a traceless linker.

FIG. 9 shows results of experiments showing that in vitro T cell proliferation showed improved immunostimulation with a traceless linker. Compared to the non-cleavable linker, the traceless linker enhanced the ability of a protein-core SNA as described herein to stimulate T-cell proliferation.

FIG. 10 shows results of experiments demonstrating that immunostimulatory protein-core SNAs held stronger potency in T cell activation. SDEC protein SNAs induced stronger CD8+ T cell proliferation compared to a simple mixture and liposomal SNAs.

FIG. 11 shows that SDEC SNAs stimulated a higher proportion of splenocytes in vivo to be CD8+ and resulted in more activated CD8+ cells.

FIG. 12 shows results of experiments designed to evaluate the memory response (CD8, Gr1+ cells) in vivo.

FIG. 13 shows the OVAp memory response as determined by flow cytometry: CD8, Gr1+.

FIG. 14 shows that SDEC SNAs activated CD4+ cells to a higher extent in mouse splenocytes in vivo.

FIG. 15 shows results of experiments designed to evaluate the memory response (Cd4, Gr1+ cells) in vivo.

FIG. 16 shows the OVAp memory response as determined by flow cytometry: CD4, Gr1+.

FIG. 17 shows that SDEC IP-SNAs induce higher proportion of memory CD8+ T cells in mouse splenocytes in vivo.

FIG. 18 shows results of an in vivo study demonstrating that the highest memory response was seen with high DNA density and traceless linker.

FIG. 19 shows results of an in vivo memory response study (CD62L−, CD44+ cells).

FIG. 20 shows results of experiments designed to evaluate OVAp memory response (CD62L−, CD44+ cells) via flow cytometry.

FIG. 21 shows results of experiments designed to evaluate the memory response (CD19− B cells) in vivo.

FIG. 22 shows the OVAp memory response as determined by flow cytometry: CD19− B cells.

FIG. 23 demonstrates that the SNA structure (amount of non-immunostimulatory strands) altered the proportion of activated and memory CD8+ T cells in mouse splenocytes in vivo.

FIG. 24 shows that the traceless linker and the surface oligonucleotide density increased proportion of CD8+ T cells in mouse splenocytes in vivo.

FIG. 25 shows results of experiments designed to evaluate the memory response (CD4, CD8 Proportions) in vivo.

FIG. 26 shows the OVAp memory response as determined by flow cytometry: CD4, CD8 cells.

FIG. 27 shows that the traceless linker and the surface oligonucleotide density increased proportion of memory CD8+ T cells in mouse splenocytes in vivo.

FIG. 28 demonstrates that the SNA structure (amount of non-immunostimulatory strands) altered the proportion of CD8+ T cells in mouse splenocytes in vivo.

FIG. 29 shows that the SNA structure (amount of non-immunostimulatory strands) altered the proportion of memory CD8+ T cells in splenocytes in vivo.

FIG. 30 shows results of experiments designed to evaluate the memory response (CD107a+ cells) in vivo.

FIG. 31 shows the OVAp memory response as determined by flow cytometry: CD107a+.

FIG. 32 shows that the SNA structure (amount of non-immunostimulatory strands) altered the ratio of CD4+ to CD8+ T cells in mouse splenocytes in vivo.

FIG. 33 depicts results of experiments showing that SDEC protein-core SNAs of the disclosure inhibited tumor growth (E.G7 lymphoma) and prolonged mouse survival.

### DETAILED DESCRIPTION

Immunotherapeutics should minimize the use of materials that cause non-specific immune responses and excess pro-inflammation cytokine release that have plagued previous clinical trials. Additionally, it is important to target the immune response to parts of the immune system most suited to the task, for example CD8+ T cell response against cancer cells or CD4+ T cell and B cell responses against viral infection. Accordingly, the present disclosure is generally directed to immunostimulatory protein-core spherical nucleic acids (IP-SNAs) comprising a protein core and a ratio of immunostimulatory and non-immunostimulatory strands, methods of making the immunostimulatory protein-core SNAs (IP-SNAs) as well as their use.

As used in this specification and the appended claims, the singular forms “a,” “an,” and “the” include plural reference unless the context clearly dictates otherwise.

The terms “polynucleotide” and “oligonucleotide” are interchangeable as used herein.

An “immunostimulatory oligonucleotide” as used herein is an oligonucleotide that can stimulate (e.g., induce or enhance) an immune response. In various embodiments, an immunostimulatory oligonucleotide comprises a class A, class B, or class C CpG sequence for both mice CpG and human CpG. In some embodiments, an immunostimulatory oligonucleotide binds to a toll-like receptor (TLR) or a NOD-like receptor (NLR). In some embodiments, the immunostimulatory comprises a sequence that is 5′-TCC ATG ACG TTC CTG ACG TT-3′ (SEQ ID NO: 9) (CpG 1826 for mouse). In some embodiments, the immunostimulatory comprises a sequence that is 5′-TCGTCGTTTTGTCGTTTTGTCGTT-3′ (CpG 7909 for human) (SEQ ID: NO: 13).

A “non-immunostimulatory oligonucleotide” as used herein is an oligonucleotide that does not stimulate (e.g., induce or enhance) an immune response on its own. In various embodiments, the non-immunostimulatory oligonucleotide comprises a sequence that is 5′-TTTTTTTTTTTTTTTTTTTT-3′ (SEQ ID NO: 5), 5′-(GGT)₇-3′ (SEQ ID NO: 6), 5′-AAAAAAAAAAAAAAAAAAAA-3′ (SEQ ID NO: 7), or 5′-(AAT)₇-3′ (SEQ ID NO: 8). In any of the embodiments or aspects of the disclosure, a non-immunostimulatory oligonucleotide does not stimulate an immune response when administered to a subject (e.g., human) at a dose of about or less than about 0.1 mg/kg.

A “linker” as used herein is a moiety that joins an oligonucleotide to a protein core of a protein-core spherical nucleic acid (SNA), as described herein. In any of the aspects or embodiments of the disclosure, a linker is a cleavable linker, a non-cleavable linker, a traceless linker, or a combination thereof.

A “subject” is a vertebrate organism. The subject can be a non-human mammal (e.g., a mouse, a rat, or a non-human primate), or the subject can be a human subject.

An “antigenic composition” is a composition suitable for administration to a human or animal subject that is capable of eliciting a specific immune response, e.g., against an antigen. Thus, an antigenic composition includes one or more antigens (for example, a tumor associated antigen, a tumor specific antigen, a neo antigen, a viral antigen) or antigenic epitopes. In some embodiments, antigenic compositions are administered to elicit an immune response that protects the subject against symptoms or conditions induced by an antigen. In certain embodiments, the antigenic composition induces or boosts an immune response against cancer. In some embodiments, symptoms or disease caused by an antigen of the disclosure is prevented, reduced, or ameliorated by inhibiting expansion of cells associated with, e.g., a tumor. In some embodiments, symptoms or disease caused by an antigen of the disclosure is prevented, reduced, or ameliorated by inhibiting replication of a virus.

An “antigen” is a molecule or molecular structure within a protein (such as may be present at the outside of a pathogen). The presence of antigens in the body could be recognized by antigen presenting cells (APCs) and normally triggers an immune response.

An “immune response” is a response of a cell of the immune system, such as a B cell or T cell, to a stimulus, such as an antigen of the disclosure (e.g., formulated as an antigenic composition or a vaccine). An immune response can be a B cell response, which results in the production of antigen-specific antibodies. An immune response can also be a T cell response, such as a CD4⁺ T cell response or a CD8⁺ T cell response. As described herein, an immune response may be an enhanced CD4+ and/or CD8+ T cell response (relative to the CD4+ and/or CD8+ T cell response in the absence of exposure to a protein-core SNA of the disclosure) depending on the protein-core SNA that is utilized (e.g., administered). An immune response can be measured, for example, by an enzyme linked immunosorbent assay (ELISA), by T cell proliferation, or by measurement of the proportion of T cell subsets such as memory or activated cells.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

### Immunostimulatory Protein-Core Spherical Nucleic Acids (IP-SNAs)

The disclosure generally provides immunostimulatory protein-core spherical nucleic acids (IP-SNAs), methods of their synthesis and methods of their use. As described herein, an IP-SNA is a structure comprising a shell of oligonucleotides arranged radially around a protein core. The spherical architecture of the oligonucleotide shell confers unique advantages over traditional nucleic acid delivery methods, including entry into nearly all cells independent of transfection agents and resistance to nuclease degradation. Protein/oligonucleotide core-shell nanoparticles are generally described in U.S. Patent Application Publication No. 2017/0232109, which is incorporated by reference herein in its entirety.

The immunostimulatory protein-core spherical nucleic acids (IP-SNAs) of the disclosure can range in size from about 1 nanometer (nm) to about 500 nm, about 1 nm to about 400 nm about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further aspects, the disclosure provides a plurality of immunostimulatory protein-core spherical nucleic acids (IP-SNAs), each immunostimulatory protein-core spherical nucleic acid (IP-SNA) comprising a shell of oligonucleotides attached thereto. In these aspects, the size of the plurality of protein-core spherical nucleic acids is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of protein-core spherical nucleic acids) of the protein-core spherical nucleic acids is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the protein-core spherical nucleic acids, for example, the amount of surface area to which oligonucleotides may be attached as described herein. It will be understood that the foregoing diameters of immunostimulatory protein-core spherical nucleic acids (IP-SNAs) can apply to the diameter of the protein-core itself or to the diameter of the protein-core and the shell of oligonucleotides associated therewith.

Protein-Core. A “protein-core” as used herein is a protein that contains one or more antigenic sequences. Thus, a protein as disclosed herein generally functions as the “core” of the immunostimulatory protein-core SNA (IP-SNA). A protein is a molecule comprising one or more polymers of amino acids. In various embodiments of the disclosure, a protein-core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically.

Proteins are understood in the art and may be either naturally occurring or non-naturally occurring. In any of the aspects or embodiments of the disclosure, a protein comprises one or more antigenic sequences.

### Naturally Occurring Proteins

Naturally occurring proteins include without limitation biologically active proteins that exist in nature or can be produced in a form that is found in nature by, for example, chemical synthesis or recombinant expression techniques. Naturally occurring proteins also include lipoproteins and post-translationally modified proteins, such as, for example and without limitation, glycosylated proteins.

### Non-Naturally Occurring Proteins

Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “peptide” typically refers to short polypeptides/proteins.

Non-naturally occurring proteins are prepared, for example, using an automated protein synthesizer or, alternatively, using recombinant expression techniques using a modified polynucleotide which encodes the desired protein.

As used herein a “fragment” of a protein is meant to refer to any portion of a protein smaller than the full-length protein or protein expression product.

As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it.

As used herein a “variant” refers to a protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art. In various aspects, proteins are modified by glycosylation, pegylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic. By way of example, an endothelial growth factor mimetic is a peptide or protein that has a biological activity comparable to the native endothelial growth factor. The term further includes peptides or proteins that indirectly mimic the activity of a protein of interest, such as by potentiating the effects of the natural ligand of the protein of interest.

In any of the aspects or embodiments of the disclosure, the protein comprises an antigen. In various embodiments, the antigen is a tumor associated antigen, a tumor specific antigen, a viral antigen, a neoantigen, or a combination thereof. In further embodiments, the antigen is OVA1, MSLN, p53, Ras, a melanoma related antigen, a HPV-related antigen, a prostate cancer related antigen, an ovarian cancer related antigen, a breast cancer related antigen, a hepatocellular carcinoma related antigen, a bowel cancer related antigen, a lung cancer related antigen, an osteocarcinoma related antigen, human papillomavirus (HPV) E6/E7 nuclear protein, or a combination thereof.

Oligonucleotides. The terms “polynucleotide” and “oligonucleotide” are used interchangeably herein. Oligonucleotides are contemplated by the present disclosure to include DNA, RNA, modified forms and combinations thereof as defined herein. Oligonucleotides may be single-stranded or double-stranded. Accordingly, in some aspects, the immunostimulatory protein-core SNA comprises DNA. In some embodiments, the DNA is double stranded, and in further embodiments the DNA is single stranded. In further aspects, the immunostimulatory protein-core SNA comprises RNA, and in still further aspects the immunostimulatory protein-core SNA comprises double stranded RNA, and in some embodiments, the double stranded RNA is a small interfering RNA (siRNA). The term “RNA” includes duplexes of two separate strands, as well as single stranded structures. Single stranded RNA also includes RNA with secondary structure. In some embodiments, RNA having a hairpin loop in contemplated. In some embodiments, the DNA is antisense DNA. Thus, in various aspects, the immunostimulatory protein-core SNA comprises DNA (single-stranded, double-stranded, or a combination thereof), RNA (single-stranded, double-stranded, or a combination thereof), modified forms and combinations thereof.

The disclosure generally provides immunostimulatory protein-core SNAs comprising a protein core and a shell of oligonucleotides attached to the protein core, wherein the shell of oligonucleotides comprises a ratio of immunostimulatory oligonucleotides to non-immunostimulatory oligonucleotides. In any of the aspects or embodiments of the disclosure, one terminus (i.e., 5′ terminus or 3′ terminus) of each oligonucleotide in the shell of oligonucleotides is attached to the protein core and the other terminus is free from any attachment. In some embodiments, when a double-stranded oligonucleotide is attached to the protein core, only one of the two oligonucleotide strands is attached to the protein core at either its 5′ or 3′ terminus. In some embodiments, when a double-stranded oligonucleotide is attached to the protein core, both of the oligonucleotide strands are attached to the protein core. In various embodiments, the ratio of immunostimulatory oligonucleotides to non-immunostimulatory oligonucleotides is between about 100:1 and 1:100. It is disclosed herein that the ratio of immunostimulatory oligonucleotides to non-immunostimulatory oligonucleotides is useful, in any of the aspects or embodiments of the disclosure, to stimulate a CD8+ T-cell response or to stimulate a CD4+ T-cell response. By “stimulate” is meant that a particular T-cell response (e.g., a CD8+ T-cell response) is increased relative to the T-cell response in the absence of exposure to a protein-core SNA of the disclosure. CD8+ and CD4+ T cell responses may be measured by methods known in the art including, for example, flow cytometry, ELISA and/or ELISPOT.

In some aspects, the disclosure provides an immunostimulatory protein-core SNA comprising a shell of oligonucleotides having a ratio of immunostimulatory oligonucleotides to non-immunostimulatory oligonucleotides that is greater than 1 is utilized (e.g., administered) for any disease or disorder that is best addressed through stimulation of a CD4+ T-cell response. Thus, in some embodiments, the ratio of immunostimulatory oligonucleotides to non-immunostimulatory oligonucleotides is, is about, or is at least about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, or more. In further embodiments, the ratio of immunostimulatory oligonucleotides to non-immunostimulatory oligonucleotides is greater than 1 and less than about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, or 50. Diseases and disorders that are best addressed through stimulation of a CD4+ T-cell response include, for example and without limitation, infections, immunodeficiency diseases, viral infections, and a combination thereof. In various embodiments, the viral infection is influenza, HIV, pneumonia virus, human papilloma virus (HPV), viral-derived cancer, or a combination thereof. In some embodiments, the ratio of immunostimulatory oligonucleotides to non-immunostimulatory oligonucleotides is about 1:0. In some embodiments, the ratio of immunostimulatory oligonucleotides to non-immunostimulatory oligonucleotides is about 100:1. In some embodiments, the ratio of immunostimulatory oligonucleotides to non-immunostimulatory oligonucleotides is about 2:1.

In some aspects, the disclosure provides an immunostimulatory protein-core SNA comprising a shell of oligonucleotides having a ratio of immunostimulatory oligonucleotides to non-immunostimulatory oligonucleotides that is less than or equal to 1 is utilized (e.g., administered) for any disease or disorder that is best addressed through stimulation of a CD8+ T-cell response. Thus, in some embodiments, the ratio of immunostimulatory oligonucleotides to non-immunostimulatory oligonucleotides is, is about, or is less than about 0.9, 0.8, 0.7, 0.6, 0.5, 0.1, 0.05, 0.01, 0.001, 0.0001, or less. In further embodiments, the ratio of immunostimulatory oligonucleotides to non-immunostimulatory oligonucleotides is less than or equal to 1 and greater than about 0.0001, 0.001, 0.01, 0.05, 0.1, 0.5, 0.6, 0.7, 0.8, or 0.9. Diseases and disorders that are best addressed through stimulation of a CD8+ T-cell response include, for example and without limitation, cancer. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the cancer is not a viral-derived cancer. In some embodiments, the ratio of immunostimulatory oligonucleotides to non-immunostimulatory oligonucleotides is about 1:1. In some embodiments, the ratio of immunostimulatory oligonucleotides to non-immunostimulatory oligonucleotides is about 2:5.

In some aspects, a subject is in need of both a protein-core comprising a shell of oligonucleotides having a ratio of immunostimulatory oligonucleotides to non-immunostimulatory oligonucleotides that is less than or equal to 1 and an immunostimulatory protein-core SNA comprising a shell of oligonucleotides having a ratio of immunostimulatory oligonucleotides to non-immunostimulatory oligonucleotides that is greater than 1. Such a subject, for example and without limitation, is a subject having both an infection and cancer.

In various embodiments, the ratio of immunostimulatory oligonucleotides to non-immunostimulatory oligonucleotides is between about 100:1 and 1:100, or between about 100:1 and 1:50, or between about 100:1 and 1:40, or between about 100:1 and 1:30, or between about 100:1 and 1:20, or between about 100:1 and 1:10, or between about 100:1 and 1:1, or between about 50:1 and 1:50, or between about 50:1 and 1:40, or between about 50:1 and 1:30, or between about 50:1 and 1:20, or between about 50:1 and 1:10, or between about 50:1 and 1:1, or between about 50:1 and 1:100, or between about 40:1 and 1:100, or between about 30:1 and 1:100, or between about 20:1 and 1:100, or between about 20:1 and 1:100, or between about 10:1 and 1:100, or between about 1:1 and 1:100. In further embodiments, the ratio of immunostimulatory oligonucleotides to non-immunostimulatory oligonucleotides is between about 1:1 and 10:1, or between about 1:1 and 9:1, or between about 1:1 and 8:1, or between about 1:1 and 7:1, or between about 1:1 and 6:1, or between about 1:1 and 5:1, or between about 1:1 and 4:1, or between about 1:1 and 3:1, or between about 1:1 and 2:1. In further embodiments, the ratio of immunostimulatory oligonucleotides to non-immunostimulatory oligonucleotides is between about 1:1 and 1:10, or between about 1:1 and 1:9, or between about 1:1 and 1:8, or between about 1:1 and 1:7, or between about 1:1 and 1:6, or between about 1:1 and 1:5, or between about 1:1 and 1:4, or between about 1:1 and 1:3, or between about 1:1 and 1:2. In some embodiments, the ratio of immunostimulatory oligonucleotides to non-immunostimulatory oligonucleotides is 1:1. In some embodiments, the ratio of immunostimulatory oligonucleotides to non-immunostimulatory oligonucleotides is 2:5. In some embodiments, the ratio of immunostimulatory oligonucleotides to non-immunostimulatory oligonucleotides is 2:4. In some embodiments, the ratio of immunostimulatory oligonucleotides to non-immunostimulatory oligonucleotides is 2:3. In some embodiments, the ratio of immunostimulatory oligonucleotides to non-immunostimulatory oligonucleotides is 1:0. In some embodiments, the ratio of immunostimulatory oligonucleotides to non-immunostimulatory oligonucleotides is 100:1. In any of the aspects or embodiments of the disclosure, the ratio of immunostimulatory oligonucleotides to non-immunostimulatory oligonucleotides is not 1:0. In some embodiments, the ratio of immunostimulatory oligonucleotides to non-immunostimulatory oligonucleotides is 2:1. In further embodiments, the ratio of immunostimulatory oligonucleotides to non-immunostimulatory oligonucleotides is about 100:1, about 50:1, about 40:1, about 30:1, about 20:1, about 10:1, about 8:1, about 7:1, about 6:1, about 5:1, about 4:1, about 3:1, about 2:1, about 1:100, about 1:50, about 1:40, about 1:30, about 1:20, about 1:10, about 1:9, about 1:8, about 1:7, about 1:6, about 1:5, about 1:4, about 1:3, about 1:2, or about 1:1.

The core-shell nanoparticle comprises, in various embodiments, a plurality of polynucleotides comprised of a sequence that is sufficiently complementary to a target sequence of a target polynucleotide such that hybridization of the polynucleotide that is part of the core-shell nanoparticle and the target polynucleotide takes place. The polynucleotide in various aspects is single stranded or double stranded, as long as the double stranded molecule also includes a single strand sequence that hybridizes to a single strand sequence of the target polynucleotide. In some aspects, hybridization of the polynucleotide that is part of the core-shell nanoparticle can form a triplex structure with a double-stranded target polynucleotide. In another aspect, a triplex structure can be formed by hybridization of a double-stranded polynucleotide that is part of a core-shell nanoparticle to a single-stranded target polynucleotide. Further description of triplex polynucleotide complexes is found in PCT/US2006/40124, which is incorporated herein by reference in its entirety.

In some embodiments, an oligonucleotide comprises a spacer as described herein.

The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotides or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Immunostimulatory protein-core SNAs are provided to which a shell of oligonucleotides is attached. Each oligonucleotide of the shell of oligonucleotides, or a modified form thereof, is generally about 5 nucleotides to about 100 nucleotides in length. More specifically, protein-core SNAs of the disclosure comprise a shell of oligonucleotide attached thereto, wherein a given oligonucleotide in the shell is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated. It will be understood that each oligonucleotide in the shell of oligonucleotides may be a different length, or some or all of the oligonucleotides in the shell of oligonucleotides may all be the same length.

In some embodiments, one or more oligonucleotides in the shell of oligonucleotides attached to a protein-core is DNA. When DNA is attached to the protein-core, the DNA is in some embodiments comprised of a sequence that is sufficiently complementary to a target region of a polynucleotide such that hybridization of the DNA polynucleotide attached to a protein-core and the target polynucleotide takes place, thereby associating the target polynucleotide to the protein-core. The DNA in various aspects is single stranded or double-stranded, as long as the double-stranded molecule also includes a single strand region that hybridizes to a single strand region of the target polynucleotide. In some aspects, hybridization of the polynucleotide attached to the protein-core can form a triplex structure with a double-stranded target polynucleotide. In another aspect, a triplex structure can be formed by hybridization of a double-stranded oligonucleotide attached to the protein-core to a single-stranded target polynucleotide. In some embodiments, the disclosure contemplates that a polynucleotide attached to a protein-core is RNA. The RNA can be either single-stranded or double-stranded (e.g., siRNA), so long as it is able to hybridize to a target polynucleotide to, e.g., inhibit expression of the target polynucleotide.

The disclosure contemplates, in various aspects, and embodiments, an immunostimulatory protein-core SNA in which the shell of oligonucleotides comprises oligonucleotides each having the same sequence, while in some aspects one or more oligonucleotides in the shell of oligonucleotides have a different sequence. In further aspects, multiple oligonucleotides in the shell of oligonucleotides are arranged in tandem and are separated by a spacer.

Spacers. In some aspects and embodiments, one or more oligonucleotides in the shell of oligonucleotides that is attached to the protein core of an IP-SNA comprise a spacer. “Spacer” as used herein means a moiety that serves to increase distance between the protein-core and the oligonucleotide, or to increase distance between individual oligonucleotides when attached to the protein-core in multiple copies, or to improve the synthesis of the IP-SNA. Thus, spacers are contemplated being located between an oligonucleotide and the protein core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the protein-core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

Immunostimulatory Protein-core SNA surface density. Generally, a surface density of oligonucleotides that is at least about 2 pmoles/cm² will be adequate to provide a stable IP-SNA. In some aspects, the surface density is at least 15 pmoles/cm². Methods are also provided wherein the oligonucleotide is attached to the protein-core of the IP-SNA at a surface density of about 2 pmol/cm² to about 200 pmol/cm², or about 10 pmol/cm² to about 100 pmol/cm². In further embodiments, the surface density is at least about 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. In further embodiments, the surface density is less than about 2 pmol/cm², less than about 3 pmol/cm², less than about 4 pmol/cm², less than about 5 pmol/cm², less than about 6 pmol/cm², less than about 7 pmol/cm², less than about 8 pmol/cm², less than about 9 pmol/cm², less than about 10 pmol/cm², less than about 15 pmol/cm², less than about 19 pmol/cm², less than about 20 pmol/cm², less than about 25 pmol/cm², less than about 30 pmol/cm², less than about 35 pmol/cm², less than about 40 pmol/cm², less than about 45 pmol/cm², less than about 50 pmol/cm², less than about 55 pmol/cm², less than about 60 pmol/cm², less than about 65 pmol/cm², less than about 70 pmol/cm², less than about 75 pmol/cm², less than about t 80 pmol/cm², less than about 85 pmol/cm², less than about 90 pmol/cm², less than about 95 pmol/cm², less than about 100 pmol/cm², less than about 125 pmol/cm², less than about 150 pmol/cm², less than about 175 pmol/cm², less than about 200 pmol/cm², less than about 250 pmol/cm², less than about 300 pmol/cm², less than about 350 pmol/cm², less than about 400 pmol/cm², less than about 450 pmol/cm², less than about 500 pmol/cm², less than about 550 pmol/cm², less than about 600 pmol/cm², less than about 650 pmol/cm², less than about 700 pmol/cm², less than about 750 pmol/cm², less than about 800 pmol/cm², less than about 850 pmol/cm², less than about 900 pmol/cm², less than about 950 pmol/cm², or less than about 1000 pmol/cm².

Alternatively, the density of oligonucleotide attached to the IP-SNA is measured by the number of oligonucleotides attached to the IP-SNA. With respect to the surface density of oligonucleotides attached to an IP-SNA of the disclosure, it is contemplated that a protein-core SNA as described herein comprises about 1 to about 2,500, or about 1 to about 500 oligonucleotides on its surface. In various embodiments, an IP-SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides in the shell of oligonucleotides attached to the protein-core. In some embodiments, an IP-SNA comprises about 80 to about 140 oligonucleotides in the shell of oligonucleotides attached to the protein-core. In further embodiments, an IP-SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the protein-core. In further embodiments, an IP-SNA consists of 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the protein-core. In still further embodiments, the shell of oligonucleotides attached to the protein-core of the IP-SNA comprises 2, 3, 4, 5, 6, 7, 8, 9, 10, 11, 12, 13, 14, 15, 16, 17, 18, 19, 20 or more oligonucleotides. In some embodiments, the shell of oligonucleotides attached to the protein-core of the IP-SNA consists of 2, 3, 4, 5, 6, 7, 8, 9, 10, 11, 12, 13, 14, 15, 16, 17, 18, 19, or 20 oligonucleotides.

### Immunostimulatory Protein-Core SNA Synthesis

The disclosure provides compositions and methods in which an oligonucleotide is associated with and/or attached to the surface of an IP-SNA via a linker. The linker can be, in various embodiments, a cleavable linker, a non-cleavable linker, a traceless linker, or a combination thereof. In some embodiments, a cleavable linker is sensitive to (and is cleaved in response to) a reducing agent (e.g., glutathione (GSH), dithiothreitol (DTT)) or a reducing environment (e.g., inside a cell). In various embodiments, a cleavable linker is sensitive to (and is cleaved in response to) various chemical stimuli such as, for example, acidity (e.g., low pH), an enzyme (e.g., peptidase), light (e.g., NIR laser), and/or hydrolysis.

The linker links the protein-core to the oligonucleotide in the disclosed protein-core SNA (i.e., protein-core-LINKER-Oligonucleotide). In various embodiments, a single oligonucleotide is attached to a linker. In further embodiments, more than one oligonucleotide (e.g., two, three, or more) is attached to a single linker. In general, linkers contemplated by the disclosure include the following, which may be used solely or in combination in the IP-SNAs of the disclosure: amide, thioether, triazole, oxime, urea, and thiourea. Some specifically contemplated linkers include carbamate alkylene, carbamate alkylenearyl dithiolate linkers, amide alkylene dithiolate linkers, amide alkylenearyl dithiolate linkers, and amide alkylene succinimidyl linkers. In some cases, the linker comprises —NH—C(O)—O—C₂₋₅alkylene-S—S—C₂₋₇alkylene- or —NH—C(O)—C₂₋₅alkylene-S—S—C₂₋₇alkylene-. The carbon alpha to the —S—S— moiety can be branched, e.g., —C(XA)(XB)—S—S— or —S—S—C(YA)(YB)- or a combination thereof, where XA, XB, YA and YB are independently H, Me, Et, or iPr. The carbon alpha to the antigen can be branched, e.g., —C(XA)(XB)-C₂₋₄alkylene-S—S—, where XA and XB are H, Me, Et, or iPr. In some cases, the linker is —NH—C(O)—O—CH₂—Ar—S—S—C₂₋₇alkylene-, and Ar is a meta- or para-substituted phenyl. In some cases, the linker is —NH—C(O)— C₂₋₄alkylene-N-succinimidyl-S—C₂₋₆alkylene-.

Additional linkers contemplated by the disclosure include those described in International Patent Publication No. WO 2018/213585, incorporated herein by reference in its entirety. In some embodiments, the linker is an SH linker, SM linker, SE linker, or SI linker. The disclosure contemplates multiple points of attachment for oligonucleotides on a protein-core.

An oligonucleotide of the disclosure may be modified at either the 5′ terminus or the 3′ terminus for attachment to a protein core.

An oligonucleotide of the disclosure can be modified at a terminus with an alkyne moiety, e.g., a DBCO-type moiety for reaction with the azide of the protein surface:

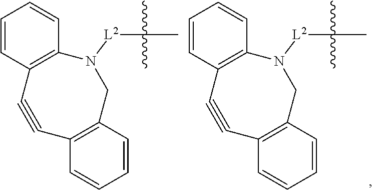

where L is a linker to a terminus of the polynucleotide. L² can be C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; wherein each Y is independently selected from the group consisting of a bond, C(O), O, NH, C(O)NH, and NHC(O); and m is 0, 1, 2, 3, 4, or 5. For example, the DBCO functional group can be attached via a linker having a structure of

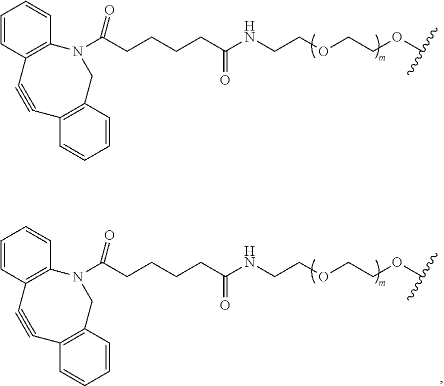

where the terminal “O” is from a terminal nucleotide on the polynucleotide. Use of this DBCO-type moiety results in a structure between the polynucleotide and the protein, in cases where a surface amine is modified, of

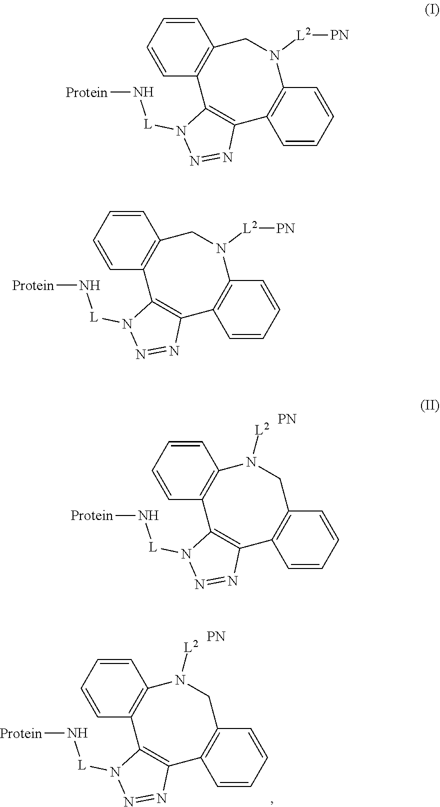

where L and L² are each independently selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and PN is the polynucleotide. Similar structures where a surface thiol or surface carboxylate of the protein are modified can be made in a similar fashion to result in comparable linkage structures.

The protein can be modified at a surface functional group (e.g., a surface amine, a surface carboxylate, a surface thiol) with a linker that terminates with an azide functional group: Protein-X-L-N₃, X is from a surface amino group (e.g., —NH—), carboxylic group (e.g., —C(O)— or —C(O)O—), or thiol group (e.g., —S—) on the protein; L is selected from C₁₋₁₀ alkylene, —Y—C(O)—C₁₋₁₀ alkylene-Y—, and —Y—C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); and m is 0, 1, 2, 3, 4, or 5. Introduction of the “L-N₃” functional group to the surface moiety of the protein can be accomplished using well-known techniques. For example, a surface amine of the protein can be reacted with an activated ester of a linker having a terminal N₃ to form an amide bond between the amine of the protein and the carboxylate of the activated ester of the linker reagent.

The oligonucleotide can be modified to include an alkyne functional group at a terminus of the oligonucleotide: Oligonucleotide-L₂-X—≡—R; L² is selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and X is a bond and R is H or C₁₋₁₀alkyl; or X and R together with the carbons to which they are attached form a 8-10 membered carbocyclic or 8-10 membered heterocyclic group. In some cases, the polynucleotide has a structure

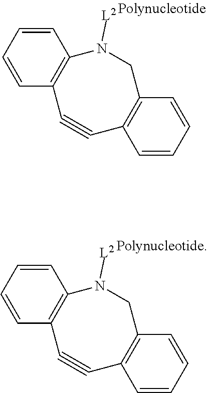

The protein, with the surface modified azide, and the polynucleotide, with a terminus modified to include an alkyne, can be reacted together to form a triazole ring in the presence of a copper (II) salt and a reducing agent to generate a copper (I) salt in situ. In some cases, a copper (I) salt is directly added. Contemplated reducing agents include ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl)phosphine, hydroquinone, and mixtures thereof.

The surface functional group of the protein can be attached to the oligonucleotide using other attachment chemistries. For example, a surface amine can be directly conjugated to a carboxylate or activated ester at a terminus of the oligonucleotide, to form an amide bond. A surface carboxylate can be conjugated to an amine on a terminus of the oligonucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the polynucleotide to form a disulfide bond. Alternatively, the thiol can be conjugated with an activated ester on a terminus of a polynucleotide to form a thiocarboxylate. Alternatively, the thiol can be conjugated with a Michael acceptor (e.g., a succinimide) on a terminus of a polynucleotide to form a thioether.

A general, representative procedure for synthesizing immunostimulatory protein-core SNAs (IP-SNAs) comprising various ratios of immunostimulatory and non-immunostimulatory oligonucleotides includes first attaching a desired amount of one type of strand (e.g., immunostimulatory) to the surface of the protein followed by the addition of the other strand to the desired ratio of the two strands. Attachment of either strand is performed by iterating over a two-step process: (1) attachment of linker to the surface of the protein and purification; (2) attachment of oligonucleotide (e.g., DNA) to the protein-conjugated linkers and purification. These two steps are repeated until a desired amount of the first strand is attached to the protein and repeated once more until the desired amount of the second strand is attached to the protein. The ratio of oligonucleotides on the surface of the protein is changed by repeating the cycle more or fewer times or using more or fewer equivalents of linkers or DNA at each step. The number of strands attached to a protein can be quantified, for example, when the protein and all but one of the oligonucleotides are labeled with unique fluorophores, using the ratio of fluorophore absorptions corrected for their respective extinction coefficients. It will be understood that the foregoing procedure is exemplary in nature.

### Uses of Immunostimulatory Protein-Core SNAs

The disclosure also includes methods of treating, reducing the symptoms of, or ameliorating a disease in a subject comprising administering to the subject an effective amount of a protein-core SNA of the disclosure (e.g., administered as a composition, pharmaceutical formulation, or antigenic composition), thereby treating the disease in the subject. Diseases or disorders that are contemplated by the disclosure in such methods include, but are not limited to, cancer, viral infections, infections, and immunodeficiency diseases.

In some embodiments, an IP-SNA of the disclosure comprising a ratio of immunostimulatory to non-immunostimulatory oligonucleotides that is less than 1 is used to stimulate a CD8+ T cell response against a cancer using an antigen associated with cancer as the IP-SNA core. In some embodiments, an IP-SNA of the disclosure comprising a ratio of immunostimulatory to non-immunostimulatory oligonucleotides that is greater than 1 may be used to stimulate a CD4+ T cell response against a viral pathogen using a viral antigen as the IP-SNA core.

In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the cancer is not viral-derived cancer. In some embodiments, the viral infection is influenza, HIV, pneumonia virus, human papilloma virus (HPV), or viral-derived cancer.

In various embodiments, the infection is Anthrax, Chickenpox, Common cold, Diphtheria, E. coli infection, Giardiasis, HIV/AIDS, Infectious, mononucleosis, Influenza (flu), Lyme disease, Malaria, Measles, Meningitis, Mumps, Poliomyelitis (polio), Pneumonia, Rocky mountain spotted fever, Rubella (German measles), Salmonella infections, Severe acute respiratory syndrome (SARS), Sexually transmitted diseases, Shingles (herpes zoster), Tetanus, Toxic shock syndrome, Tuberculosis, Viral hepatitis, West Nile virus, Whooping cough (pertussis).

In various embodiments, the immunodeficiency disease is Immunodeficiency diseases includes but not limited to ataxia-telangiectasia, chediak-Higashi syndrome, combined immunodeficiency disease, complement deficiencies, DiGeorge syndrome, hypogammaglobulinemia, Job syndrome, leukocyte adhesion defects, panhypogamma globulinemia, Bruton's disease, congenital agammaglobulinemia, selective deficiency of IgA, Wiskott-Aldrich syndrome.

### Uses of IP-SNAs in Gene Regulation/Therapy

In some aspects, an IP-SNA as disclosed herein possesses the ability to regulate gene expression. Thus, in some embodiments, an IP-SNA of the disclosure comprises an oligonucleotide having gene regulatory activity (e.g., inhibition of target gene expression). Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of an IP-SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of IP-SNA and a specific oligonucleotide.

In various aspects, the methods include use of an oligonucleotide which is 100% complementary to a target polynucleotide, i.e., a perfect match, while in other aspects, the oligonucleotide is about or at least (meaning greater than or equal to) about 95% complementary to the polynucleotide over the length of the oligonucleotide, about or at least about 90%, about or at least about 85%, about or at least about 80%, about or at least about 75%, about or at least about 70%, about or at least about 65%, about or at least about 60%, about or at least about 55%, about or at least about 50%, about or at least about 45%, about or at least about 40%, about or at least about 35%, about or at least about 30%, about or at least about 25%, about or at least about 20% complementary to the polynucleotide over the length of the oligonucleotide to the extent that the oligonucleotide is able to achieve the desired degree of inhibition of a target gene product. Moreover, an oligonucleotide may hybridize over one or more segments such that intervening or adjacent segments are not involved in the hybridization event (e.g., a loop structure or hairpin structure). The percent complementarity is determined over the length of the oligonucleotide. For example, given an inhibitory oligonucleotide in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et aL., J. Mol. Biol., 1990, 215, 403-410; Zhang and Madden, Genome Res., 1997, 7, 649-656).

Accordingly, methods of utilizing an IP-SNA of the disclosure in gene regulation therapy are provided. This method comprises the step of hybridizing a polynucleotide encoding a target gene with one or more oligonucleotides complementary to all or a portion of the polynucleotide, the oligonucleotide being attached to or associated with an IP-SNA as described herein, wherein hybridizing between the polynucleotide and the oligonucleotide occurs over a length of the polynucleotide with a degree of complementarity sufficient to inhibit expression of the target gene product. The inhibition of gene expression may occur in vivo or in vitro. The oligonucleotide may be either RNA or DNA. The RNA can be an inhibitory RNA (RNAi) that performs a regulatory function, and in various embodiments is selected from the group consisting of a small inhibitory RNA (siRNA), an RNA that forms a triplex with double stranded DNA, and a ribozyme. Alternatively, the RNA is microRNA that performs a regulatory function. The DNA is, in some embodiments, an antisense-DNA.

### Compositions

The disclosure also provides compositions that comprise a protein-core SNA of the disclosure, or a plurality thereof. In some embodiments, the composition is an antigenic composition. In some embodiments, the composition further comprises a pharmaceutically acceptable carrier. The term “carrier” refers to a vehicle within which the protein-core SNA as described herein is administered to a subject. Any conventional media or agent that is compatible with the protein-core SNAs according to the disclosure can be used. The term carrier encompasses diluents, excipients, adjuvants and a combination thereof. Pharmaceutically acceptable carriers are well known in the art (see, e.g., Remington's Pharmaceutical Sciences by Martin, 1975, the entire disclosure of which is herein incorporated by reference).

Exemplary “diluents” include water for injection, saline solution, buffers such as Tris, acetates, citrates or phosphates, fixed oils, polyethylene glycols, glycerine, propylene glycol or other synthetic solvents. Exemplary “excipients” include but are not limited to stabilizers such as amino acids and amino acid derivatives, polyethylene glycols and polyethylene glycol derivatives, polyols, acids, amines, polysaccharides or polysaccharide derivatives, salts, and surfactants; and pH-adjusting agents. “Adjuvant” refers to a substance which, when added to a composition comprising an antigen, enhances or potentiates an immune response to the antigen in the recipient upon exposure. In any of the aspects or embodiments of the disclosure, the protein-core SNAs provided herein comprise immunostimulatory oligonucleotides (for example and without limitation, a CpG oligonucleotide) as adjuvants and a protein-core comprising an antigen. Other adjuvants known in the art may also be used in the compositions of the disclosure. For example, the adjuvant may be aluminum or a salt thereof, mineral oils, Freund adjuvant, vegetable oils, water-in-oil emulsion, mineral salts, small molecules (e.g., imiquimod, resiquimod), bacterial components (e.g., flagellin, monophosphoryl lipid A), or a combination thereof.

The disclosure includes methods for eliciting an immune response in a subject in need thereof, comprising administering to the subject an effective amount of an antigenic composition comprising one or more of the protein-core SNAs of the disclosure. An “effective amount” is that amount of a protein-core SNA that alone, or together with further doses, produces the desired response, e.g., elicits an immune response. Unless otherwise indicated, the antigenic composition is an immunogenic composition.

The immune response generated by the IP-SNA as disclosed herein generates an immune response that recognizes, and preferably ameliorates and/or neutralizes, a disease (e.g., cancer, an immunodeficiency disease, a viral infection) as described herein. Methods for assessing antibody responses after administration of an antigenic composition are known in the art and include, e.g., flow cytometry, T cell killing assay, ELISA, and/or EliSPOT. As described herein, immunostimulatory protein-core SNA compositions may be tailored to specifically enhance a CD8+ T cell response and/or a CD4+ T cell response.

Antigenic compositions can be administered via any suitable route, such as parenteral administration, intramuscular injection, subcutaneous injection, intradermal administration and mucosal administration such as oral or intranasal. Additional routes of administration include but are not limited to intravenous, intraperitoneal, intranasal administration, intra-vaginal, intra-rectal, and oral administration. A combination of different routes of administration, separately or at the same time, is also contemplated by the disclosure.

Administration can involve a single dose or a multiple dose schedule. The dose can be delivered once, continuously, such as by continuous pump, or at periodic intervals. The periodic interval may be weekly, bi-weekly, or monthly. The dosing can occur over the period of one month, two months, three months or more to elicit an appropriate humoral and/or cellular immune response.

In some embodiments, a composition for administration comprises a plurality of IP-SNAs, wherein the core of each IP-SNA is a different protein. In some embodiments, a composition for administration comprises a plurality of IP-SNAs, wherein the core of each IP-SNA is the same protein. In some embodiments, a composition for administration comprises a plurality of IP-SNAs, wherein the core of at least two IP-SNAs is the same protein. In some embodiments, a composition for administration comprises a plurality of IP-SNAs, wherein the composition includes one or more IP-SNAs wherein the protein core is a synthetic fusion of two or more proteins. Synthetic fusion proteins include, without limitation, an expressed fusion protein (expressed from a single gene) and post-expression fusions where proteins are conjugated together chemically.

### EXAMPLES

The following examples are provided to illustrate, but not to limit, the subject matter described herein.

The experiments detailed below led to several conclusions. Superior methods for synthesizing immunostimulatory protein-core SNAs (IP-SNAs) via an iterative synthesis process is described, wherein the methods add a few strands to the protein per cycle, increasing the total DNA density with each iteration. It was found that IP-SNAs elicit a strong immune response against the antigen which comprises the core, and the generated immune response is often superior to a simple mixture of the immunostimulatory components (antigen core and adjuvant DNA). Increasing surface-density of DNA resulted in stronger immune responses.

Similar immune responses were generated with fewer CpG strands when some CpG strands are replaced with non-immunostimulatory (“filler”) T20 strands while the total surface-density was held constant. Traceless linkage (SDEC) between the DNA and protein core enhanced the immune stimulation compared to a non-cleavable linkage (BMPS). Finally, the IP-SNA structure (e.g., the ratio of CpG to non-immunostimulatory T20 strands) can be used to modulate the type of immune response elicited (e.g., CD8+ versus CD4+ T cell response).

### Example 1

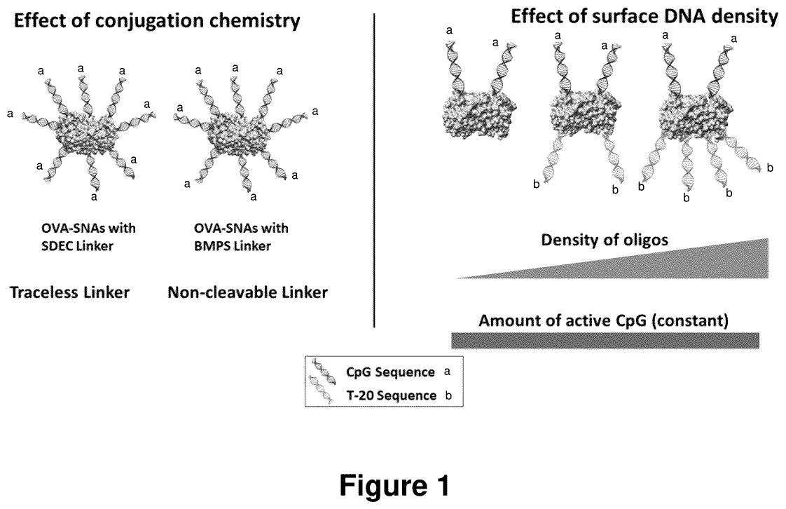

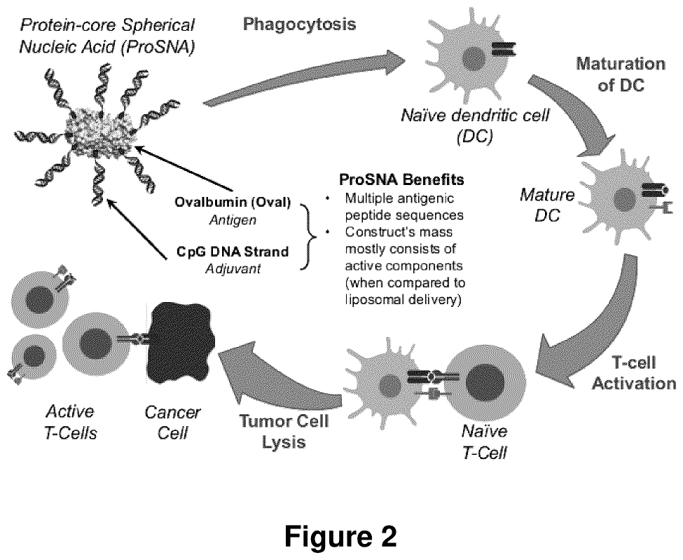

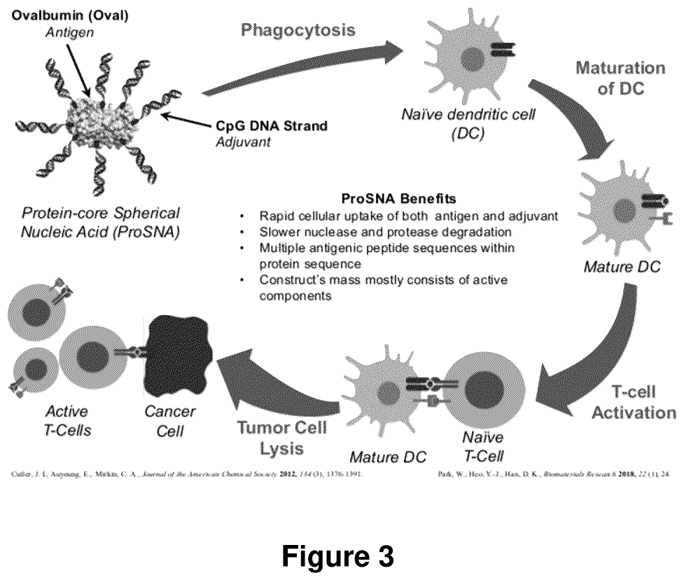

As described herein, the present disclosure is generally directed to the design of immunostimulatory protein-core spherical nucleic acids (SNAs). The disclosure provides, in various aspects, methods to synthesize immunostimulatory protein-core SNAs with immunostimulatory and non-immunostimulatory strands. Exemplary synthesized protein-core SNA structures are depicted in FIG. 1. FIG. 1 shows that the number of immunostimulatory oligonucleotides present on a protein-core SNA may be kept constant while the number of non-immunostimulatory filler strands is varied. FIG. 1 also depicts that protein-core SNAs of the disclosure may be produced, in some embodiments, using traceless linkers or non-cleavable linkers. FIG. 2 and FIG. 3 are schematics depicting the activation of T-cells and show that cancer immunotherapy requires the activation of T-cells targeted against cancer cells with an antigen and adjuvant. Benefits of protein-core SNAs as disclosed herein include but are not limited to: they provide rapid cellular uptake of both antigen and adjuvant; they demonstrate slower nuclease and protease degradation; they allow for multiple antigenic peptide sequences within a protein sequence; and the mass of the construct mostly consists of active components.

### Example 2

### IP-SNA Synthesis and Characterization

Materials. Phosphate-Buffered Saline solution (PBS) was purchased from Invitrogen at 10× concentration (pH 7.4). Pre-purified ovalbumin (Oval) in PBS was acquired from LS-Bio. All Alexafluor dyes and the non-cleavable linker (BMPS) were obtained from ThermoFisher Scientific. 4-12% sodium dodecyl sulfate polyacrylamide (SDS-PAGE) gels and the corresponding running buffer and loading dye were also obtained from ThermoFisher. Size exclusion columns were purchased from GE Healthcare (illustra NAP, Sephadex G-25 resin). All DNA synthesis reagents, including all specialty phosphoramidites (e.g., Cy3, Cy5, and fluorescein), were obtained through Glen Research. Dihydroxyacetophenone (DHAP) matrix was acquired from Fisher Scientific. Triethylammonium acetate (TEAA) pH 7 buffer, 30% methylamine solution, 40% ammonium hydroxide solution, glacial acetic acid, dithiothreitol (DTT), 2-mercaptoethanol were purchased from Sigma-Aldrich. Molecular weight cut-off filters (MWCO) were obtained from Fisher Scientific (MilliporeSigma Amicon).

Dye-Labeling Ovalbumin. Ovalbumin (Oval) in PBS was labeled with Alexa-Fluor NHS ester dye according to the manufacturer's instructions. Briefly, Oval was incubated in 1×PBS with AF dye at a 1:1 molar ratio overnight at 4° C. If aggregates and contaminants were present, they and free dye were removed using fast protein liquid chromatography (FPLC, SEC 650 column in PBS pH 7.4 buffer). Otherwise, free dye was removed from the protein-dye conjugates (Oval-AF) using size exclusion chromatography (SEC) with NAP 25 columns. The ratio of dye per ovalbumin was determined using UV-vis spectroscopy.

DNA Synthesis. A MerMade 12 Oligonucleotide synthesizer was used for synthesizing the non-dye-labeled strands while an ABI 394 was used for synthesizing the dye-labeled strands. All strands were synthesized with phosphorothioate backbones using standard phosphoramidite chemistry. Following synthesis, the strands were separated from the solid support using a 1:1 volume mixture of aqueous ammonium hydroxide (30%) and methylamine (40%) for 50 minutes at 55° C. (non-dye-labeled) or overnight at room temperature (dye-labeled). The solutions were then evaporated using a gentle stream of N₂ gas. Next, the DNA was reconstituted in TEAA pH 7 buffer and then passed through a syringe filter (0.2 μm pore size) into a separate vial prior to reverse phase high-pressure liquid chromatography (Shimadzu or Agilent; gradient of aqueous triethylammonium acetate and acetonitrile: 0-100% over 45 minutes) with a C4 column (for Cy5 labeled) or a C18 column (for non-dye-labeled). After drying the samples were dried by lyophilization, the DNA was rehydrated and treated with 20% acetic acid for one hour. The DNA was then washed three times with ethyl acetate and the aqueous layer was removed to capture the DNA. Following another lyophilization, the DNA was reconstituted in water, aliquoted, and stored at −20° C.

CpG Sequence:
(SEQ ID NO: 10)
5′- TCC ATG ACG TTC CTG ACG TT (Sp18) C3Thiol-3′
Cy5 Labeled CpG Sequence:
(SEQ ID NO: 11)
5′- TCC ATG ACG TTC CTG ACG TT (Sp18) Cy5
C3Thiol-3′
T20 Non-Adjuvant Sequence:
(SEQ ID NO: 12)
5′- TTT TTT TTT TTT TTT TTT TT (Sp18) C3Thiol-3′

DNA Reduction and Characterization. The day that DNA needed to be used in the synthesis of a protein-core SNA (ProSNA) of the disclosure, the appropriate number of aliquots were thawed at room temperature and treated with 100 mM dithiothreitol (room temperature, 1 hour). The reduced DNA was then purified with SEC (Sephadex G-25 resin) and its concentration was determined with UV-vis spectroscopy. UV-vis spectroscopy was also used to calculate the average number of Cy5 dye molecules per DNA strands. Matrix assisted laser desorption/ionization mass spectroscopy (MALDI MS), using DHAP as the matrix, was used to determine the molecular weight of the DNA to confirm its synthesis and purification.

Traceless Linker Synthesis and Workup. 2-mercaptoethanol (0.70 mL, 10.0 mmol, 1 equiv) was added dropwise to a stirring solution of 2,2′-dipyridyl disulfide (4.4 g, 20.0 mmol, 2 equiv) in 40 mL of methanol at room temperature. After stirring overnight, the methanol was removed under reduced pressure using a rotary evaporator. The 2-(2-pyridyldithio)ethanol was purified on a silica column with a gradient of DCM to methanol. A solution of the 2-(2-Pyridyldithio)ethanol and pyridine (95 μL, 1.18 mmol, 1.1 equiv) in 8 mL of anhydrous acetonitrile was added dropwise to a stirring suspension of succinimidyl carbonyl (0.41 g, 1.60 mmol, 1.5 equiv) in 5 mL of anhydrous acetonitrile at room temperature. The reaction was stirred overnight. The solvent was removed by rotary evaporator under reduced pressure and the crude reaction dissolved in ethyl acetate. The solution was extracted with saturated aqueous NaHCO₃, brine, dried over MgSO₄, filtered, and concentrated under reduced pressure. The product was precipitated with diethyl ether from a solution in a small amount of DCM, the resulting white powder was dried under vacuum.

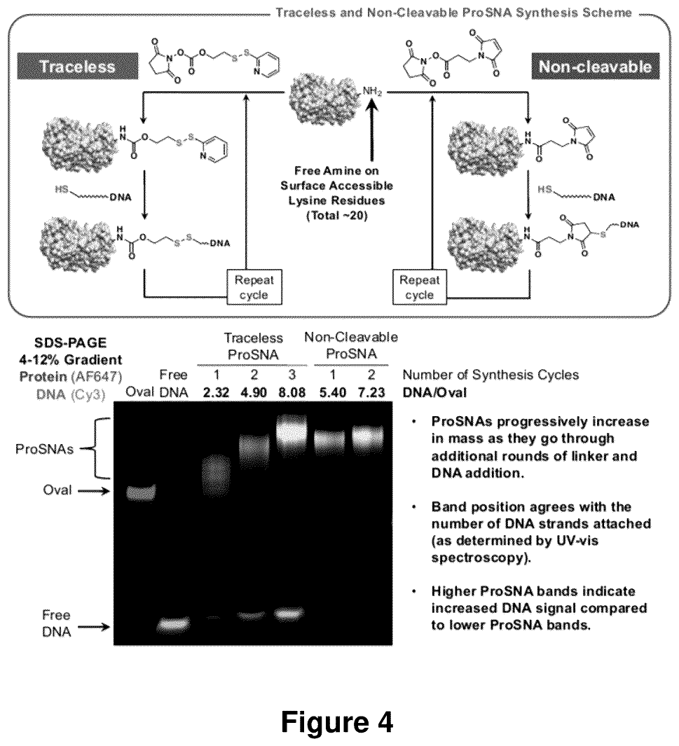

Immunostimulatory Protein-Core SNA Synthesis. First, Oval-AF was combined with linker for 2 hours at 4° C. The traceless linker (SDEC) and non-cleavable linker (BMPS) were added at a concentration slightly lower than 1.5 μM, a threshold concentration previously determined to cause protein precipitation. This reaction occurred at 4° C. for 3 hours and free linker was removed with SEC (NAP column, Sephadex G-25 resin). The reduced thiol-terminated DNA was added to the protein-linker conjugates at a ratio of approximately 10:1 and allowed to react overnight at 4° C. Free DNA was removed using a 30 kDa MWCO spin filter (8 spins at 5500 rcf for 5 minutes). The free DNA filtrate was collected and concentrated with a 3 kDa MWCO filter (1 spin at 5000 rcf for 30 minutes). This entire process was repeated to attach additional DNA strands. The concentrated free DNA that was recovered from the MWCO spins were added back to the reaction at the DNA addition step. Absorbance at 260 nm (non-labeled DNA) or 649 nm (Cy5-labeled DNA) corrected for dye-labeled protein absorbance was used to determine DNA concentration. SDS-PAGE gel electrophoresis was used to confirm ProSNA formation and were run as recommended by the provided protocol. These gels were then imaged on an Amersham Typhoon Gel Imaging System. See FIG. 4.

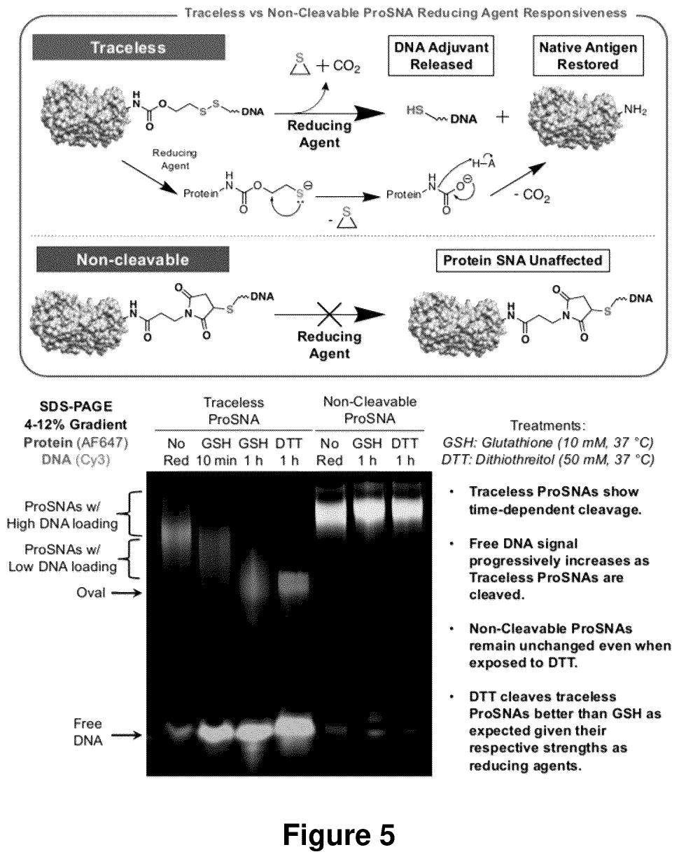

Testing ProSNA Responsiveness to Reducing Agent. Protein-core SNAs were treated with reducing agent (dithiothreitol or 2-mercaptoethanol), while ensuring that the pH of the reaction remained neutral (pH ˜7.4). The reduced samples were then run in a 4-12% SDS-PAGE gel and the gel was imaged on the Amersham Typhoon Gel Imaging System. See FIG. 5.

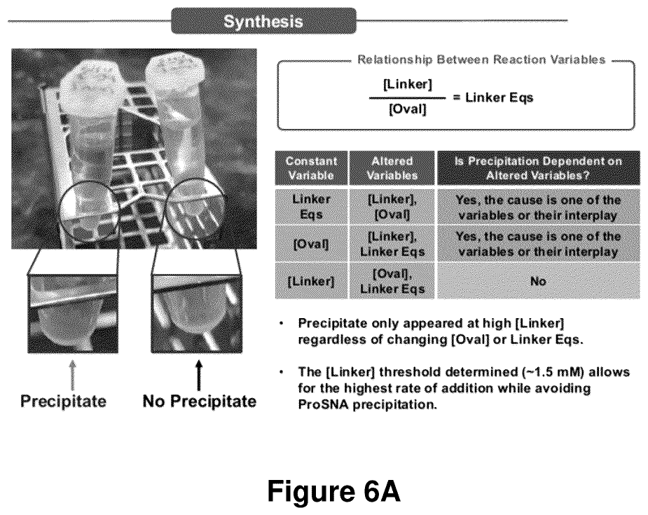

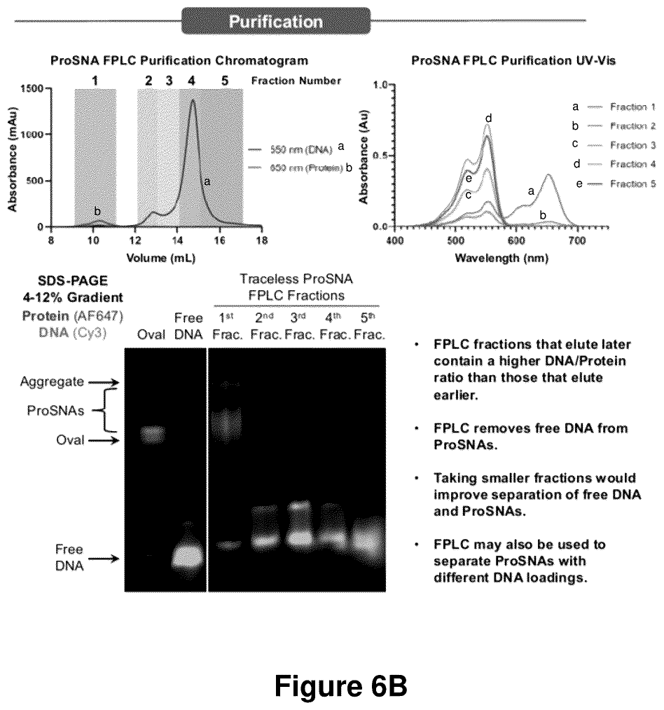

Investigation of Protein Precipitation During Traceless Linker Addition. In five separate reactions (FIGS. 6A and 6B; Table 1), the traceless linker was added to ovalbumin labeled with Alexafluor-647 (Oval-AF647) to investigate the three parameters of the reaction: linker concentration, ovalbumin concentration, and linker to ovalbumin molar ratio. The reactions ran at 4° C. for 3 hours and then were checked for precipitation. The varied parameters of the five traceless linker addition reactions run to determine the cause of protein precipitation and whether the resulting reaction caused protein precipitation.

TABLE 1
The varied parameters of the five traceless linker addition reactions
run to determine the cause of protein precipitation and whether
the resulting reaction caused protein precipitation. “Linker
equivalencies” indicate the linker to ovalbumin molar ratio.

| | Linker | | Ovalbumin | | Linker | Precipitate |
|---|---|---|---|---|---|---|
| Reaction | Concentration | | Concentration | | Equivalencies | Observed? |
| 1 | 1 | mM | 0.2 | μM | 5000 | No |
| 2 | 1 | mM | 10 | μM | 100 | No |
| 3 | 7 | mM | 1.4 | μM | 5000 | Yes |
| 4 | 7 | mM | 70 | μM | 100 | Yes |
| 5 | 1.4 | mM | 70 | μM | 20 | No |

### Assessment of IP-SNA Efficacy Using in vitro and in vivo Assays

Materials. All cell culture reagents, including sterile PBS, Roswell Park Memorial Institute 1640 medium (RPMI 1640), penicillin/streptomycin (P/S), fetal bovine serum (FBS), Live/Dead Fixable Stains, Cell Proliferation Dye eFluor 450, and trypan blue solution were obtained from ThermoFisher Scientific. 1.2 mL microtiter tubes that were used to hold samples for flow cytometry were purchased from Thermo Fisher Scientific as well. All antibodies, EliSpot materials, and mouse recombinant GM-CSF were obtained from BioLegend.

Mouse Spleen Collection and Processing. Spleens were first collected and placed in RPMI 1640 cell culture medium with 10% FBS and P/S. Next, they were mashed through 70-micron strainers, using sterile PBS to wash the strainers. The mashed spleens were pelleted by centrifugation (1400 rpm, 5 minutes, 4° C.) and the supernatant was removed. The pellet was immediately disaggregated using Ack red blood cell lysis buffer. After 2 minutes, PBS was added to halt the lysis buffer. The cells were washed again and resuspended by RPMI 1640 cell culture medium with 10% FBS and P/S before further use. The concentration of splenocytes was then determined using a Countess II Automated Cell Counter.

In vitro T-Cell Proliferation. After collecting and processing OT1 or OT2 mice spleens, the splenocytes were treated with Cell Proliferation Dye eFluor 450 as per manufacturer's protocol. Afterwards, cells were washed with cold RPMI/FBS/P/S, spun down at 1200 rpm for 5 minutes, and resuspended to a concentration of 3×10⁶ cells/mL. Cells were plated in a 96-well round bottom plate with 3E×10⁵ cells/well and treated with SNAs. After incubation at 37° C. for 72 hours, cells were stained with Live/Dead Fixable Green Stain, as per manufacturer's protocol, and 1 μg/mL of the corresponding antibodies (CD8 for OT1, CD4 for OT2). Following a short vortex, the tubes were kept in darkness at 4° C. for 20 minutes. The strength of the response was quantified through the percentage of CD8⁺ T-cells that underwent at least one division, as indicated by the dilution of the eFluor 450 dye when analyzed with FlowJo.

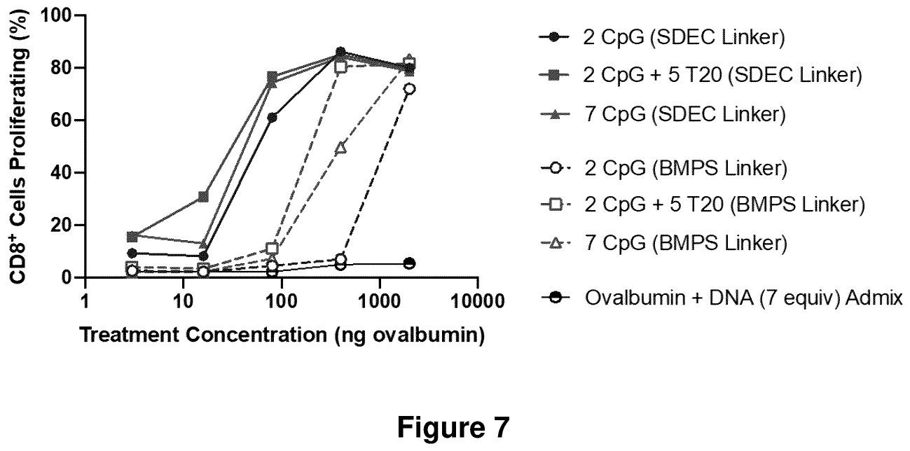

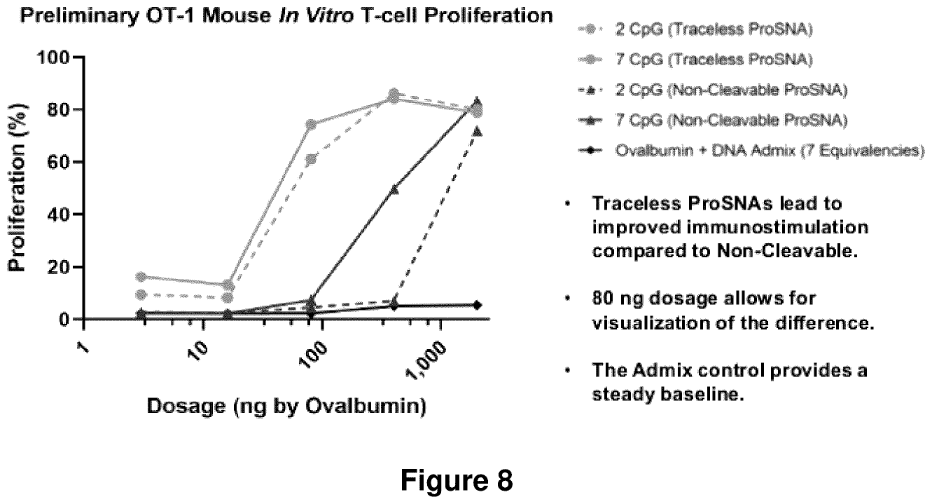

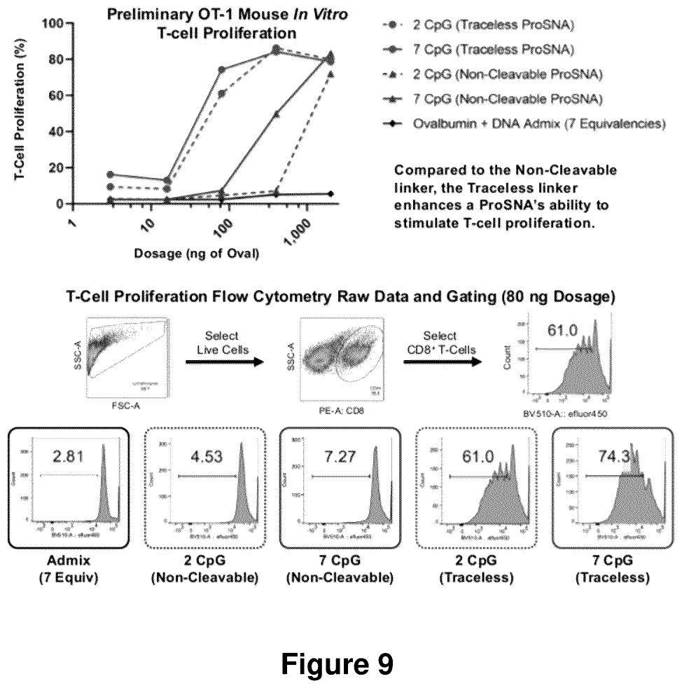

The data in FIGS. 7, 8, and 9 demonstrate that: Protein SNAs have much higher T cell proliferation efficacy compared to a simple mixture (admix, open black circles). By adding non-immunostimulatory T20 DNA strands, while holding the amount of active immunostimulatory components constant (ovalbumin antigen and CpG adjuvant), the potency of the protein SNAs improved (red solid squares versus blue solid circles). If the surface DNA density is kept constant (7 strands per protein) but the ratio of active CpG DNA to non-immunostimulatory T20 DNA (red squares versus green triangle in FIG. 7) is decreased, equivalent potency and efficacy is retained while treating with less CpG adjuvant. The cleavable and traceless SDEC linker improved the potency of protein SNAs compared to a non-cleavable BMPS for all structures studied (all solid versus all dashed lines in FIG. 7).

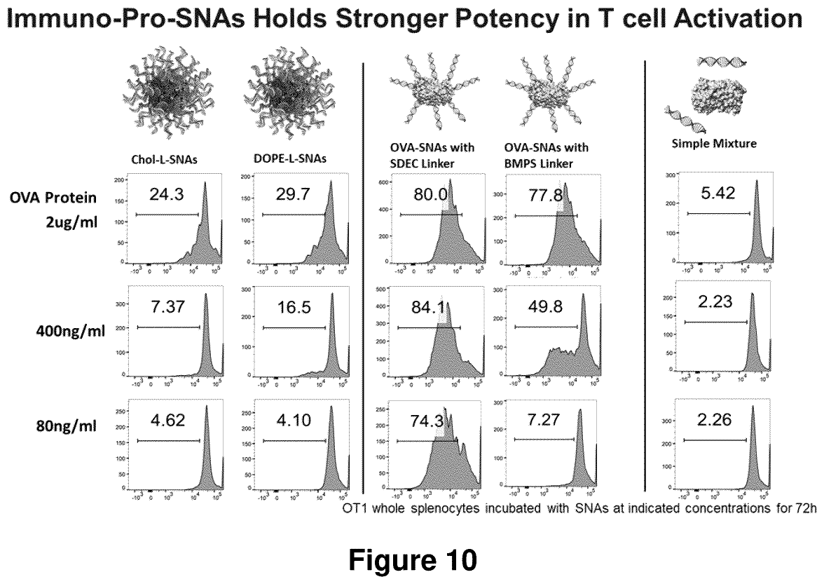

Data in FIG. 10 also compared the T cell proliferation stimulation ability from IP-SNAs and conventional liposome-core SNAs. Data shows that IP-SNAs with SDEC linkage show greater potential in stimulating CD8+ T cells in vitro, compared with liposome-core SNAs.

Memory Response Treatment Plan and Flow Cytometry. In vivo T cell memory response analyses were performed by vaccinating C57BL/6 mice (on days 0, 14, and 28) with simple mixture of adjuvant and antigen or different IP-SNAs composed of 30 ug of Oval protein. Spleens were collected on day 35 and whole splenocytes are processed as protocol described above. After determining cell concentration, flow cytometry was used to measure CD8+ T cells percentage, activated CD8+ T cell percentage (CD8+/Gr1+), as well as CD4+ T cell percentage. Memory phenotype markers (CD62L and CD44) and T cell degranulation markers (CD107a) were also measured by flow cytometry. The results were analyzed with FlowJo and Kruskal-Wallis or 2-way ANOVAs with post hoc tests were used to determine the significance of differences observed.

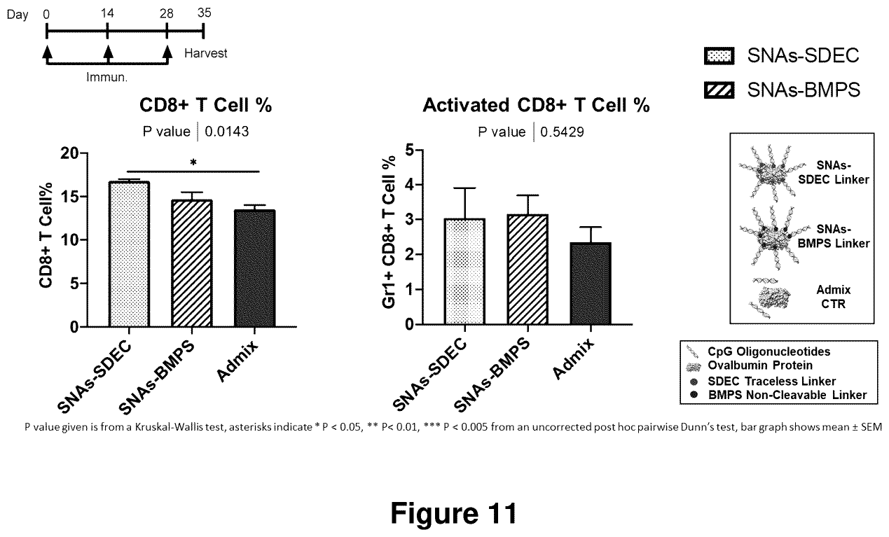

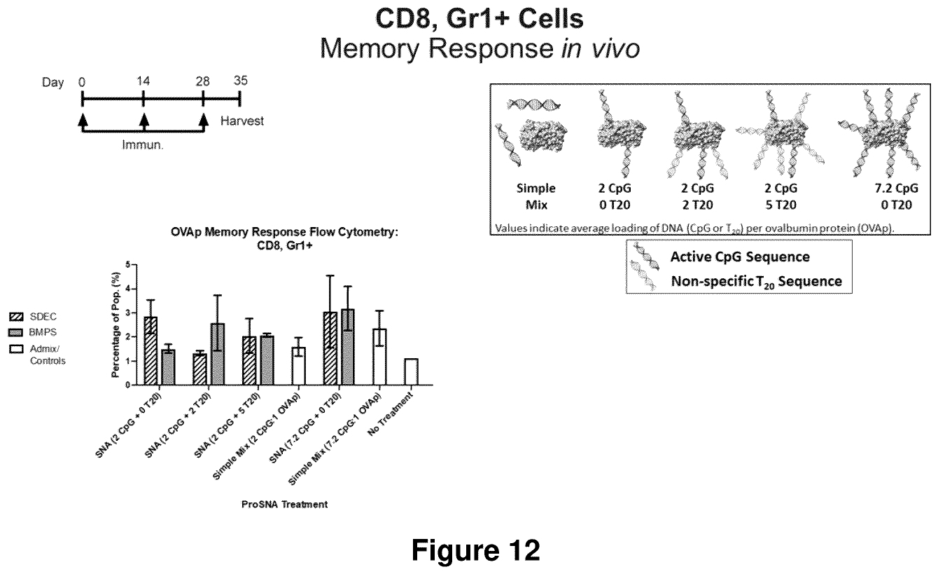

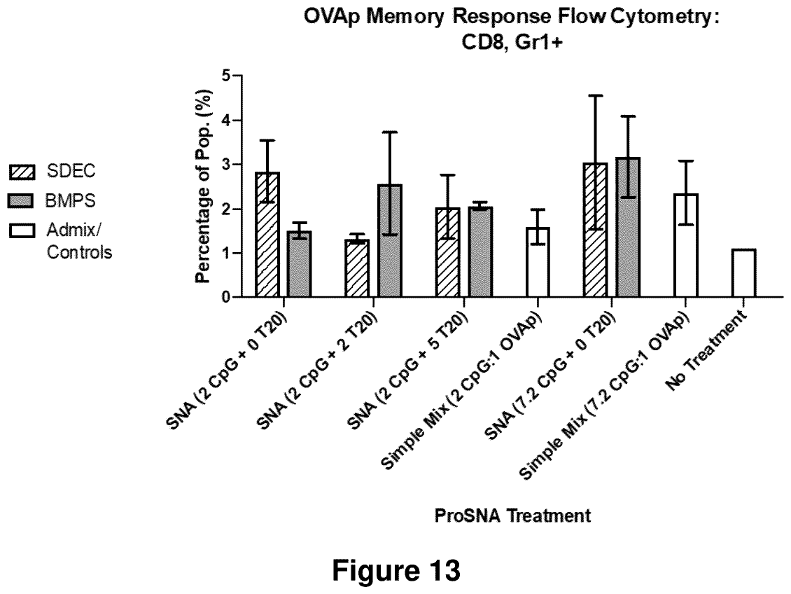

Activating CD8+ T cells is crucial in anti-tumor immune responses because activated CD8+ T cells (CTLs) display strong ability to attack and lyse cancer cells. Data in FIG. 11 (right panel) shows that IP-SNAs elicit a higher proportion of CD8+ T cells in splenocytes compared to a simple mixture (patterned bars versus gray bar). This is further supported by the result of the Kruskal-Wallis test (P=0.0143) which suggests that the proportion of all splenocytes that are CD8+ T cells varies between the treatment groups and the post hoc Dunn's test that shows a significant difference between the SNA-SDEC and simple mixture groups (P<0.05). In addition, IP-SNAs made with traceless SDEC linker (dotted bars) elicits a larger proportion of CD8+ T cells within all splenocytes compared to IP-SNAs made with non-cleavable BMPS (hashed bars). See also FIGS. 12 and 13.

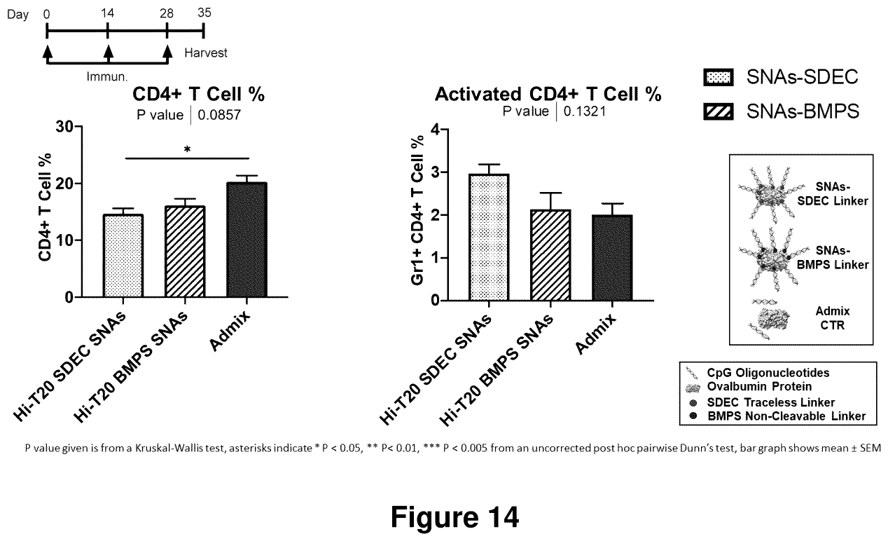

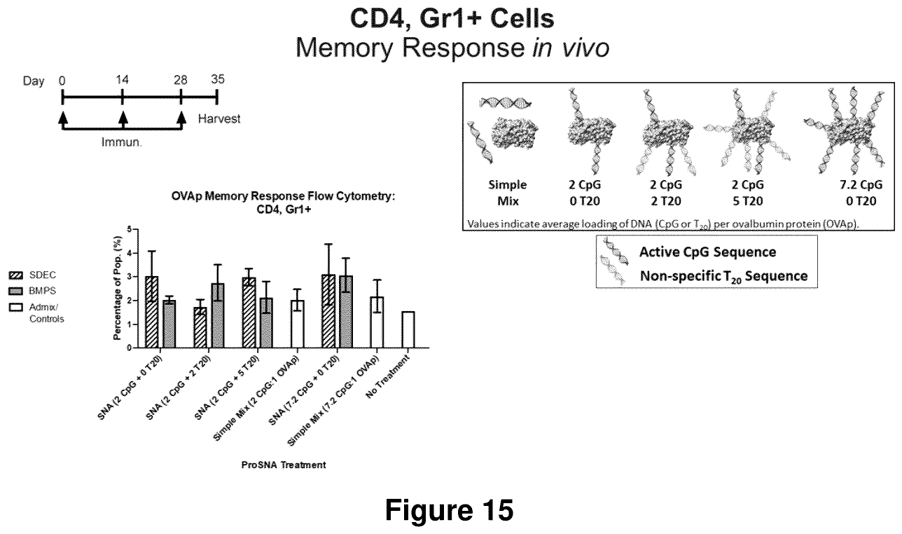

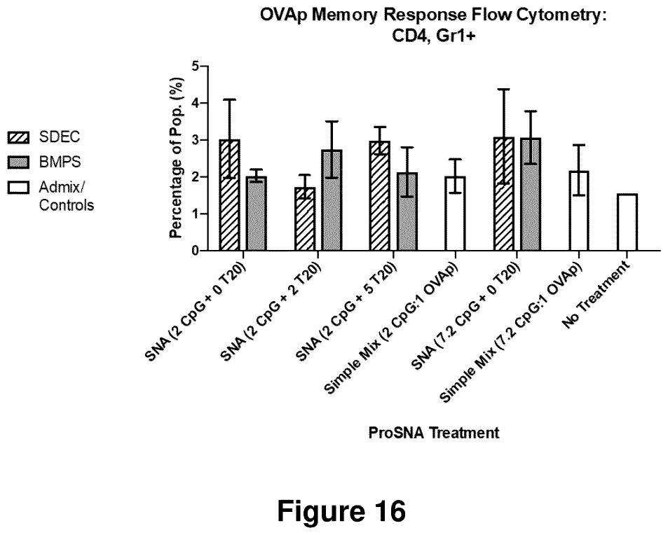

CD4+ T cell priming is critical to generate a strong antibody reaction against antigens, especially important for overcoming viral infections. Data in FIG. 14 (right panel) shows that IP-SNAs elicit a higher proportion of activated CD4+ T cells in all splenocytes compared with a simple mixture (right graph, patterned versus gray bars) and IP-SNAs made with traceless SDEC linkers (dotted pattern) generate a higher proportion of activated CD4+ T cells than IP-SNAs made with non-cleavable BMPS linkers (hashed pattern). See also FIGS. 15 and 16.

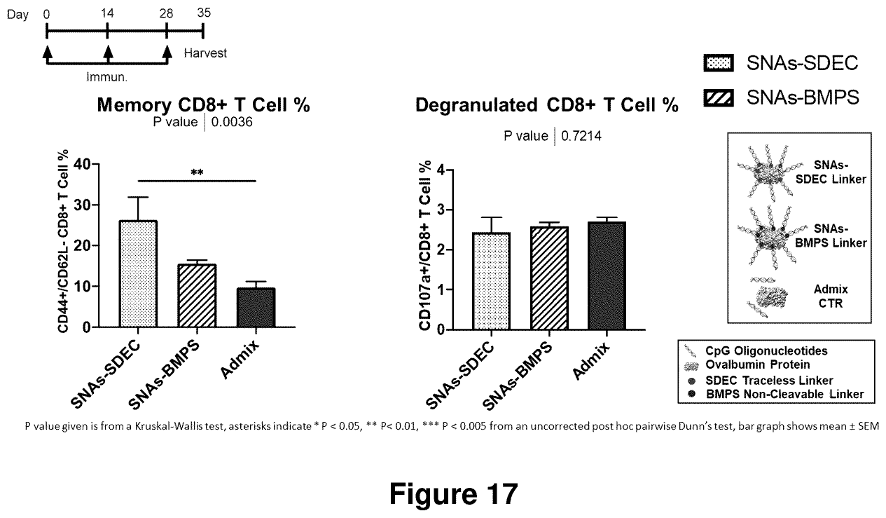

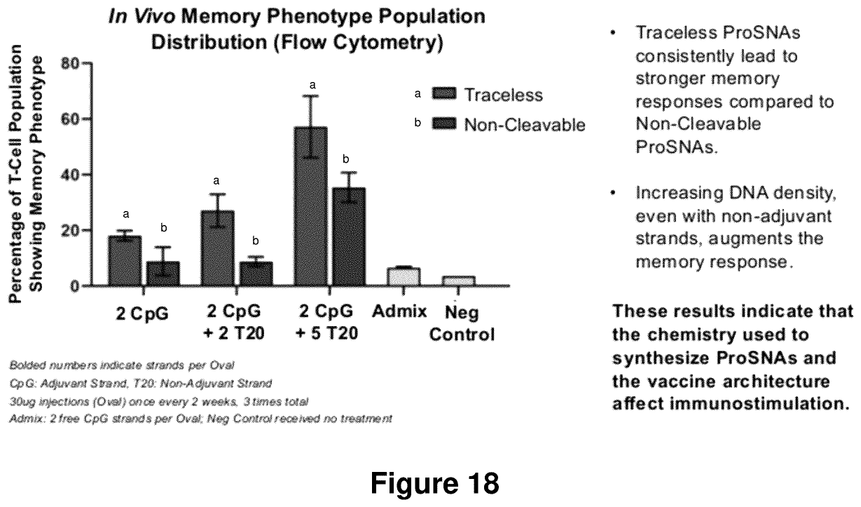

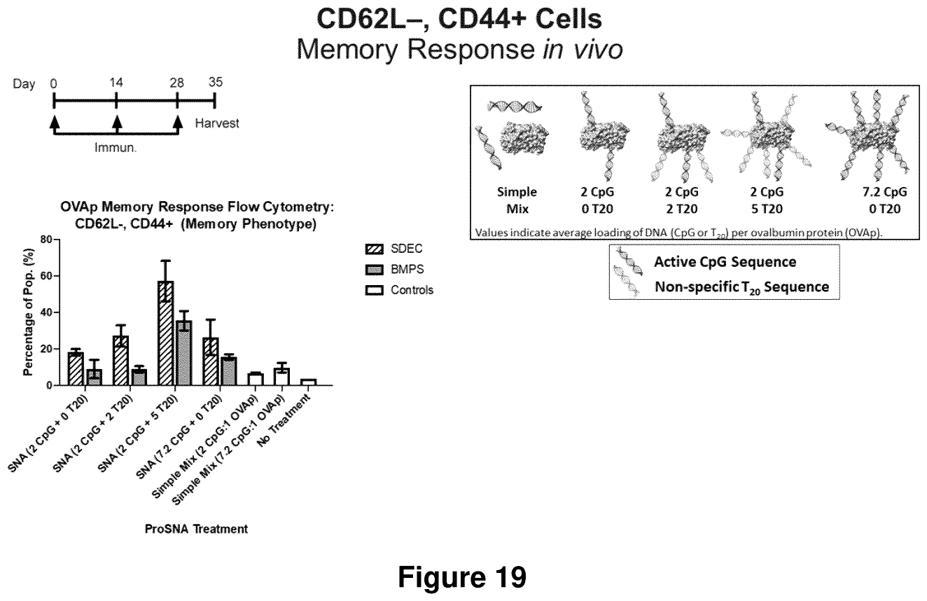

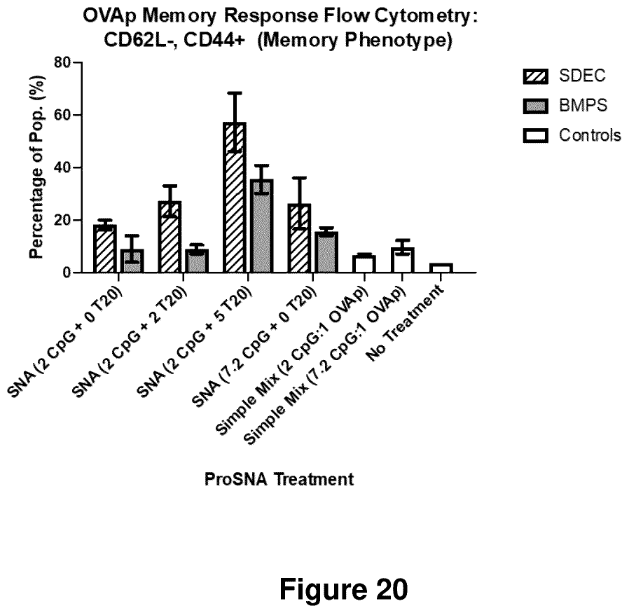

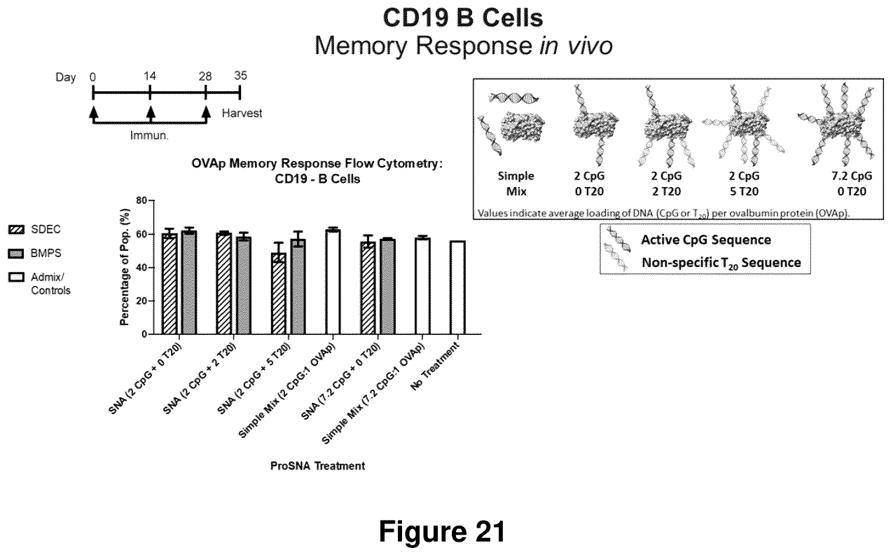

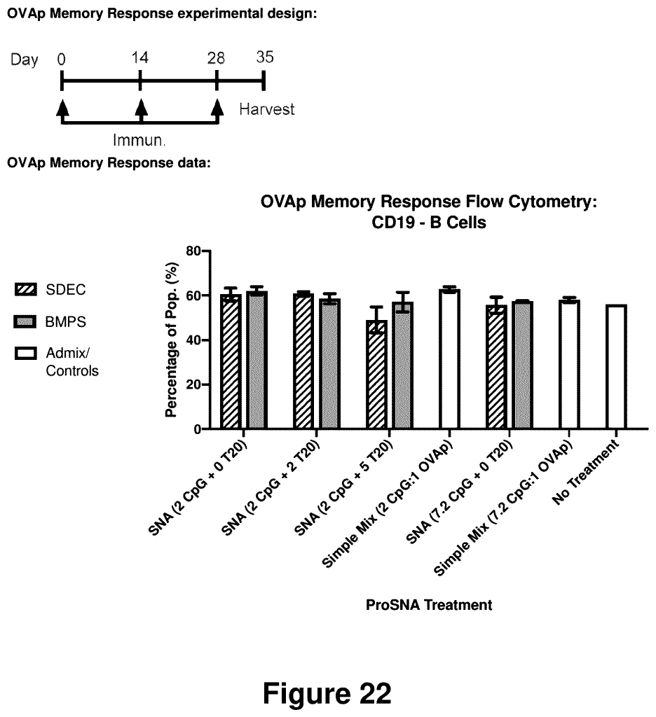

Memory phenotype T cells are critical for a long-lasting response to the antigen of interest. Analysis in FIG. 17 (left graph) shows that IP-SNAs elicit a much stronger memory CD8+ T cell response than a simple admix of antigen and adjuvant. This is supported by the Kruskal-Wallis test that shows the proportion of memory CD8+ T cells changes with the treatment (P=0.0036) and the post hoc pairwise Dunn's test between SNAs-SDEC and Admix (P<0.01). In addition, IP-SNAs made with SDEC linker (dotted pattern) display stronger memory T cell responses compared to IP-SNAs made with BMPS linker (hashed pattern). See also FIGS. 18, 19, and 20. FIGS. 21 and 22 demonstrate the CD19 B cell memory response in vivo.

To demonstrate that increased amount of surface-conjugated oligonucleotide results in stronger immune stimulation, different IP-SNAs with fixed number of CpG DNA but with an increasing number of T20 non-immunostimulatory DNA attached to their surface were also analyzed in vivo, using the same protocol described above.

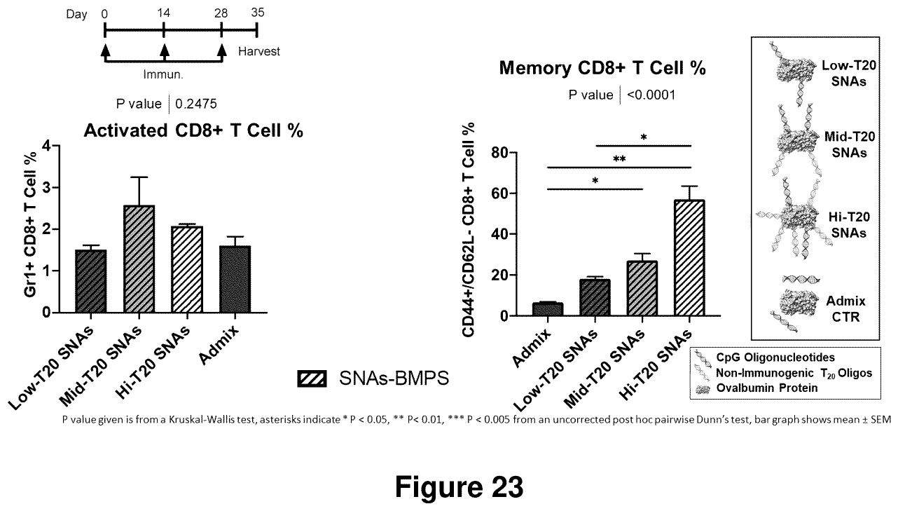

Data in FIG. 23 (right panel) shows that decreasing the ratio of adjuvant to non-immunostimulatory DNA on the surface of IP-SNAs while increasing total DNA surface density caused an increase in the percentage of activated CD8+ T cells as well as the memory CD8+ T cells, even though the amount of immunostimulatory components (protein antigen and DNA adjuvant) remained constant. This is further supported by the Kruskal-Wallis test that indicates the proportion of memory CD8+ T cells differs between the treatment groups (P<0.0001) and multiple statistically significant post hoc pairwise Dunn's test comparisons.

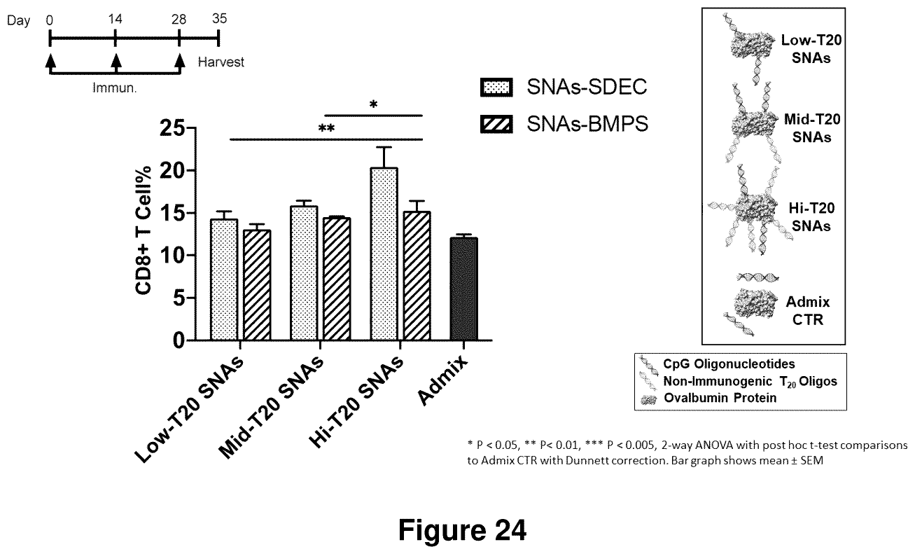

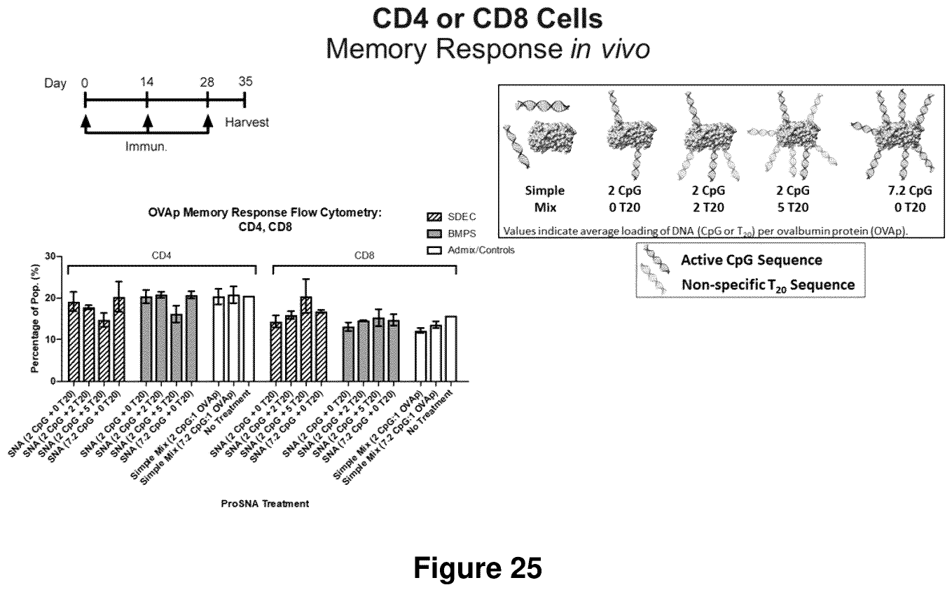

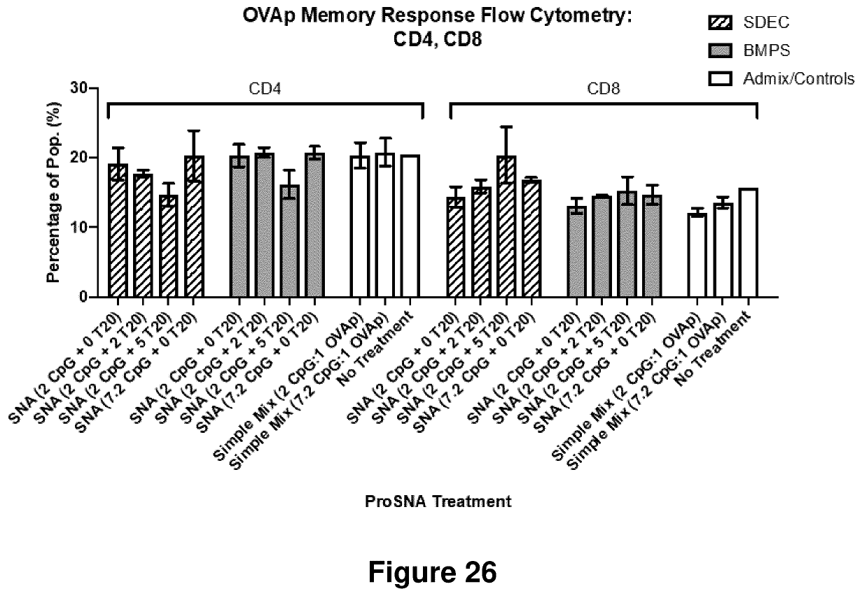

In addition, data in FIG. 24 also shows that adding non-immunostimulatory T20 DNA (increasing total DNA density and decreasing the ratio of adjuvant to T20), while holding the amount of immunostimulatory components (ovalbumin antigen and CpG adjuvant) constant across all treatments, increases the proportion of CD8+ T cells. This is further supported by 2-way ANOVA analysis that indicates this ratio is a significant source of variation (P=0.0136). See also FIGS. 25 and 26.

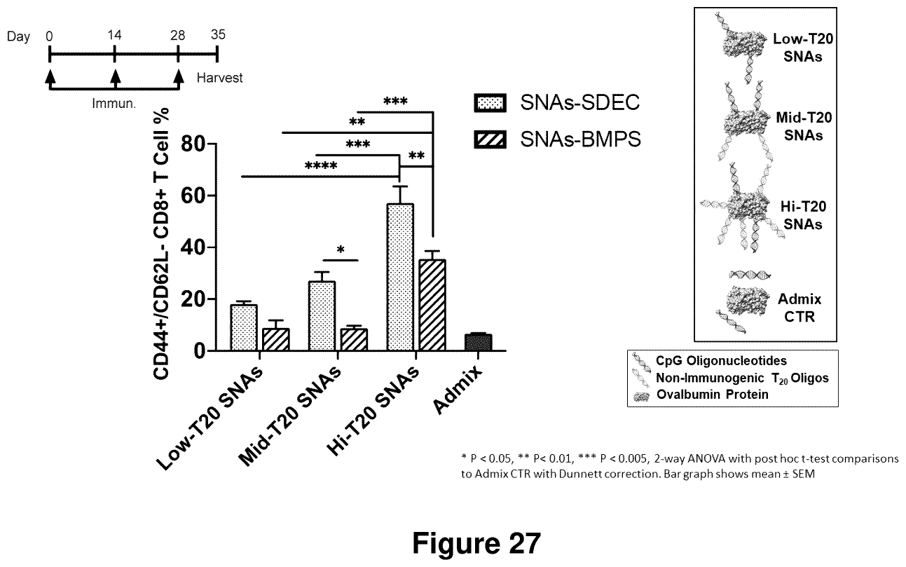

Data in FIG. 27 demonstrates the consistent trend by showing the effects of different IP-SNAs on CD8+ T percentages (scheme on the right). By adding more and more non-immunostimulatory sequence (T20), memory CD8+ T cell percentage could be increased, even if all of the immuno-components remained the same (Protein antigen core and CpG as adjuvant) (scheme on the left). This is further supported by 2-way ANOVA analysis that indicates the amount of filler strand is a significant source of variation (P<0.0001).

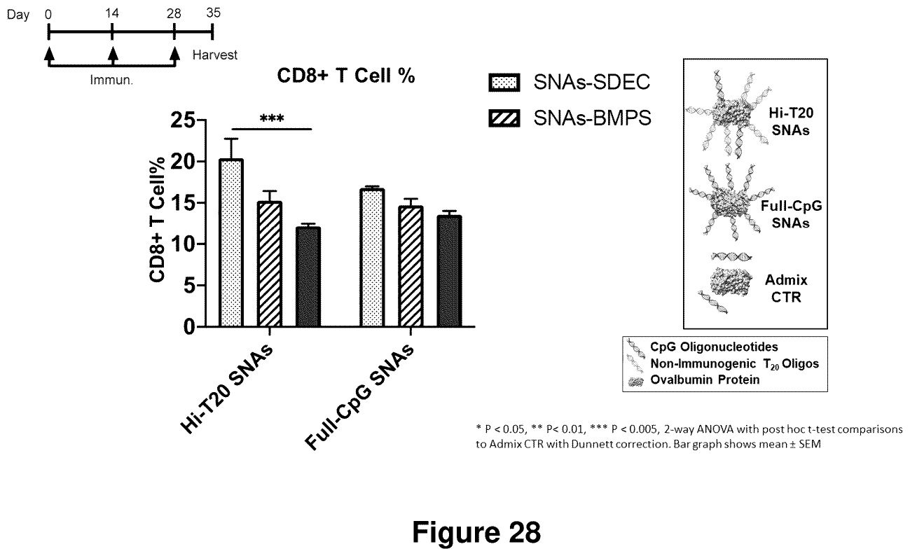

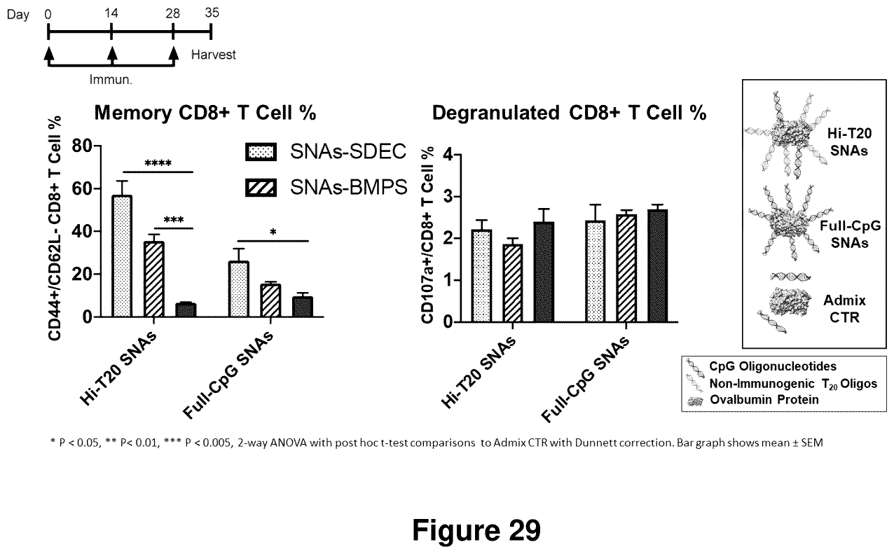

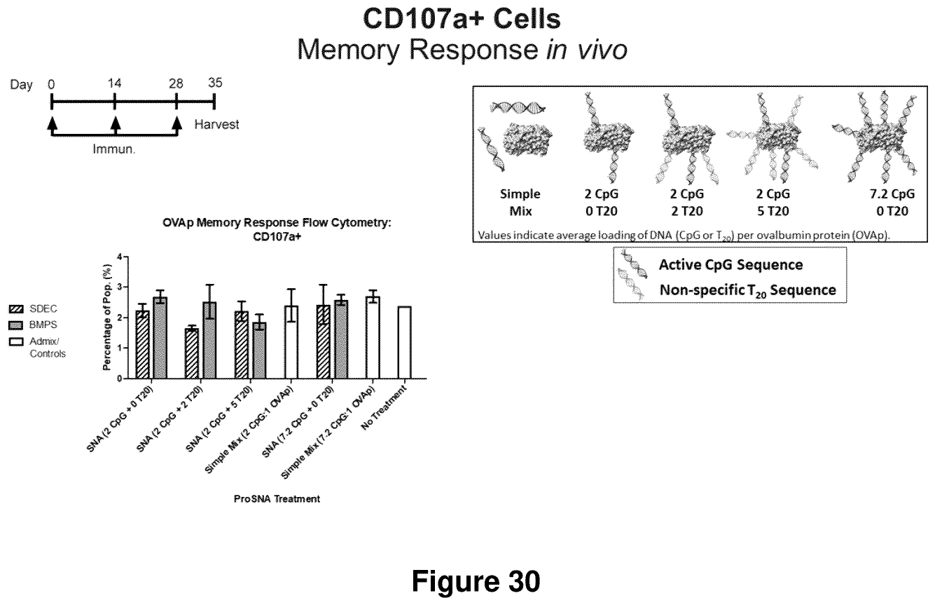

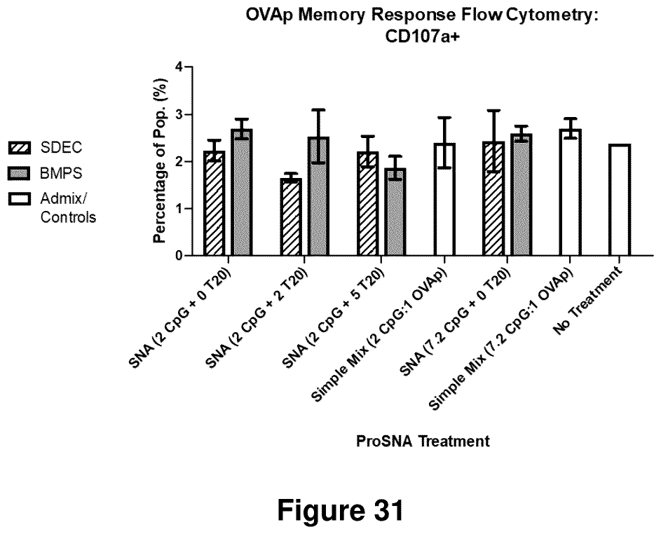

To prove that similar immune stimulation is generated with fewer CpG strands by using non-immunostimulatory filler strands, IP-SNAs with 7 CpG DNA conjugated to the protein were compared to IP-SNAs with same oligonucleotides surface density but lower CpG ratio (2 CpG and 5 T20 non-immunostimulatory DNA). Data in FIG. 28 shows that IP-SNAs with equivalent total DNA density, but lower ratio of adjuvant to non-immunostimulatory strands, result in equivalent or higher proportion of CD8+ T cells (left bars versus right bars of any single pattern). FIG. 29 further proves this trend by showing the percent of memory CD8+ T cells is increased when a lower ratio of adjuvant to non-immunostimulatory strand are present on the surface of IP-SNAs (left graph, left group of bars versus right group of bars). This is further supported by 2-Way ANOVA analysis that indicates that animals treated with SNAs bearing different ratios of CpG to T20 strands result in differing memory CD8+ T cell percentages (P=0.0020). See also FIGS. 30 and 31.

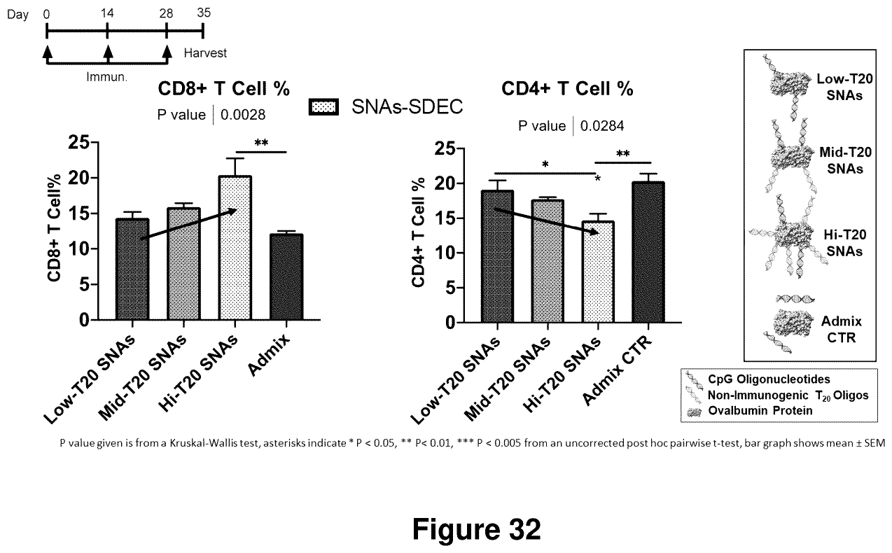

Finally, FIG. 32 demonstrates that the protein SNA structure (ratio of CpG to non-immunostimulatory strands) can be used to modulate CD8+ versus CD4+ T cell responses Left panel shows that by increasing the density of total DNAs on the surface, CD8+ T cell percentage could be enhanced; right panel shows the opposite trend, by decreasing the total surface DNAs density on IP-SNAs, CD4+ T cell percentage could be enhanced.

Prophylactic In Vivo Vaccination with IP-SNAs Against E.G7-OVA Tumor

Mice were immunized with different IP-SNAs (SDEC linkage, 7 CpG DNA) or PBS, 19 days and 5 days before the inoculation of tumor cells (2×10⁵ EG.7-OVA cells) into the right flank of C57BL/6 mice (n=5). Tumor growth and animal survival were measured.

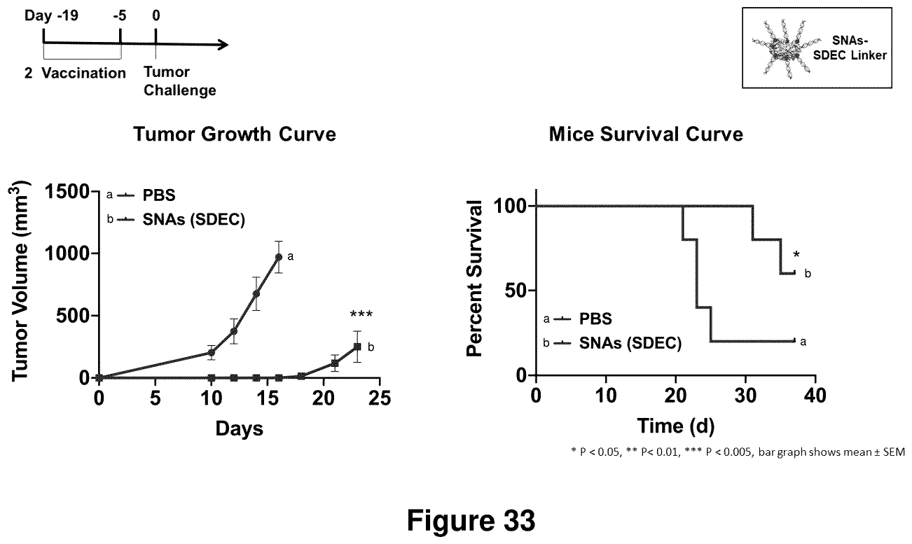

Tumor growth for all groups treated with SNAs was significantly slower than for the PBS group. Differences in tumor burden were statistically significant between IP-SNAs group and PBS group. Kaplan-Meier survival curves of different treatment groups. IP-SNAs showed significant benefit in prolonging life over PBS treatments. Statistical significance for survival analysis was calculated by the log-rank test: ***P<0.001, **P<0.01, *P<0.05. See FIG. 33.

### REFERENCES

1. Cerritelli, S.; Velluto, D.; Hubbell, J. A. PEG-SS-PPS: Reduction-Sensitive Disulfide Block Copolymer Vesicles for Intracellular Drug Delivery. Biomacromolecules 2007, 8, 1966-1972.
2. Van Der Vlies, A. J.; Oneil, C. P.; Hasegawa, U.; Hammond, N.; Hubbell, J. A. Synthesis of Pyridyl Disulfide-Functionalized Nanoparticles for Conjugating Thiol-Containing Small Molecules, Peptides, and Proteins. Bioconjug. Chem. 2010, 21 (4), 653-662.
3. Hirosue, S.; Kourtis, I. C.; van der Vlies, A. J.; Hubbell, J. A.; Swartz, M. A. Antigen Delivery to Dendritic Cells by Poly(Propylene Sulfide) Nanoparticles with Disulfide Conjugated Peptides: Cross-Presentation and T Cell Activation. Vaccine 2010, 28 (50), 7897-7906.
4. Xu, J.; Wang, J.; Luft, J. C.; Tian, S.; Owens, G.; Pandya, A. A.; Berglund, P.; Pohlhaus, P.; Maynor, B. W.; Smith, J.; et al. Rendering Protein-Based Particles Transiently Insoluble for Therapeutic Applications. J. Am. Chem. Soc. 2012, 134 (21), 8774-8777.
5. Mueller, S. N.; Tian, S.; Desimone, J. M. Rapid and Persistent Delivery of Antigen by Lymph Node Targeting PRINT Nanoparticle Vaccine Carrier to Promote Humoral Immunity. Mol. Pharm. 2015, 12 (5), 1356-1365.
6. Ma, D.; Tian, S.; Baryza, J.; Luft, J. C.; Desimone, J. M. Reductively Responsive Hydrogel Nanoparticles with Uniform Size, Shape, and Tunable Composition for Systemic Sirna Delivery in Vivo. Mol. Pharm. 2015, 12 (10), 3518-3526.
7. Kapadia, C. H.; Tian, S.; Perry, J. L.; Luft, J. C.; Desimone, J. M. Reduction Sensitive PEG Hydrogels for Codelivery of Antigen and Adjuvant to Induce Potent CTLs. Mol. Pharm. 2016, 13 (10), 3381-3394.
8. Tan, X.; Lu, X.; Jia, F.; Liu, X.; Sun, Y.; Logan, J. K.; Zhang, K. Blurring the Role of Oligonucleotides: Spherical Nucleic Acids as a Drug Delivery Vehicle. J. Am. Chem. Soc. 2016, 138, 10834-10837.
9. Suma, T.; Cui, J.; Mu, M.; Fu, S.; Tran, J.; Noi, K. F.; Ju, Y.; Caruso, F. Modulated Fragmentation of Proapoptotic Peptide Nanoparticles Regulates Cytotoxicity. J. Am. Chem. Soc. 2017, 139 (11), 4009-4018.
10. Jones, L. R.; Goun, E. A.; Shinde, R.; Rothbard, J. B.; Contag, C. H.; Wender, P. A. Releasable Luciferin Transporter Conjugates: Tools for the Real-Time Analysis of Cellular Uptake and Release. J. Am. Chem. Soc. 2006, 128 (20), 6526-6527.
11. Staben, L. R.; Koenig, S. G.; Lehar, S. M.; Vandlen, R.; Zhang, D.; Chuh, J.; Yu, S.-F.; Ng, C.; Guo, J.; Liu, Y.; et al. Targeted Drug Delivery through the Traceless Release of Tertiary and Heteroaryl Amines from Antibody-Drug Conjugates. Nat. Chem. 2016, No. October.
12. Nembrini, C.; Stano, A.; Dane, K. Y.; Ballester, M.; van der Vlies, A. J.; Marsland, B. J.; Swartz, M. A.; Hubbell, J. A. Nanoparticle Conjugation of Antigen Enhances Cytotoxic T-Cell Responses in Pulmonary Vaccination. Proc. Natl. Acad. Sci. 2011, 108 (44), E989-E997.
13. de Titta, A.; Ballester, M.; Julier, Z.; Nembrini, C.; Jeanbart, L.; van der Vlies, A. J.; Swartz, M. A.; Hubbell, J. A. Nanoparticle Conjugation of CpG Enhances Adjuvancy for Cellular Immunity and Memory Recall at Low Dose. Proc. Natl. Acad. Sci. 2013, 110 (49), 19902-19907.
14. Luo, M.; Wang, H.; Wang, Z.; Cai, H.; Lu, Z.; Li, Y.; Du, M.; Huang, G.; Wang, C.; Chen, X.; et al. A STING-Activating Nanovaccine for Cancer Immunotherapy. Nat. Nanotechnol. 2017, 12 (7), 648-654.
15. Datta, S. K.; Takabayashi, K.; Raz, E. The Therapeutic Potential of Antigen-Oligonucleotide Conjugates. Ann. N. Y. Acad. Sci. 2003, 1002, 105-111.
16. Maurer, T.; Heit, A.; Hochrein, H.; Ampenberger, F.; O'Keeffe, M.; Bauer, S.; Lipford, G. B.; Vabulas, R. M.; Wagner, H. CpG-DNA Aided Cross-Presentation of Soluble Antigens by Dendritic Cells. Eur. J. Immunol. 2002, 32 (8), 2356-2364.
17. Slutter, B.; Soema, P. C.; Ding, Z.; Verheul, R.; Hennink, W.; Jiskoot, W. Conjugation of Ovalbumin to Trimethyl Chitosan Improves Immunogenicity of the Antigen. J. Control. Release 2010, 143 (2), 207-214.
18. Herbdth, M.; Szekeres, Z.; Kovesdi, D.; Papp, K.; Erdei, A.; Prechl, J. Coadministration of Antigen-Conjugated and Free CpG: Effects of in Vitro and in Vivo Interactions in a Murine Model. Immunol. Lett. 2014, 160 (2), 178-185.
19. Kramer, K.; Shields, N. J.; Poppe, V.; Young, S. L.; Walker, G. F. Intracellular Cleavable CpG Oligodeoxynucleotide-Antigen Conjugate Enhances Anti-Tumor Immunity. Mol. Ther. 2017, 25 (1), 62-70.
20. Kramer, K.; Young, S. L.; Walker, G. F. Comparative Study of 5′- and 3′-Linked CpG-Antigen Conjugates for the Induction of Cellular Immune Responses. ACS Omega 2017, 2 (1), 227-235.
21. Heit, A.; Schmitz, F.; O'Keeffe, M.; Staib, C.; Busch, D. H.; Wagner, H.; Huster, K. M. Protective CD8 T Cell Immunity Triggered by CpG-Protein Conjugates Competes with the Efficacy of Live Vaccines. J. Immunol. 2005, 174 (7), 4373-4380.
22. Kreutz, M.; Giquel, B.; Hu, Q.; Abuknesha, R.; Uematsu, S.; Akira, S.; Nestle, F. O.; Diebold, S. S. Antibody-Antigen-Adjuvant Conjugates Enable Co-Delivery of Antigen and Adjuvant to Dendritic Cells in Cis but Only Have Partial Targeting Specificity. PLoS One 2012, 7 (7).
23. Clauson, R. M.; Berg, B.; Chertok, B. The Content of CpG-DNA in Antigen-CpG Conjugate Vaccines Determines Their Cross-Presentation Activity. Bioconjug. Chem. 2019, 30 (3), 561-567.
24. Heit, A.; Huster, K. M.; Schmitz, F.; Schiemann, M.; Busch, D. H.; Wagner, H. CpG-DNA Aided Cross-Priming by Cross-Presenting B Cells. J. Immunol. 2004, 172 (3), 1501-1507.
25. Verthelyi D., Klinman D. M. (2002) CpG-ODN-Safety Considerations. In: Raz E. (eds) Microbial DNA and Host Immunity. Humana Press, Totowa, NJ
26. Risini D. Weeratnaa, Michael J. McCluskiea, YuXua, c, Heather L. Davis. CpG DNA induces stronger immune responses with less toxicity than other adjuvants (2000) 8, 1755-1762
27. Yanal M Muradt, Timothy M Clay, H Kim Lyerly & Michael A Morse. CPG-7909 (PF-3512676, ProMune®): toll-like receptor-9 agonist in cancer therapy. (2007) Expert Opinion on Biological Therapy, 7:8, 1257-1266
28. Clive S. Zent, Brian J. Smith, Zuhair K. Ballas, James E. Wooldridge, Brian, K. Link, Timothy G. Call, Tait D. Shanafelt, Deborah A. Bowen, Neil E. Kay, Thomas E. Witzig & George J. Weiner. Phase I clinical trial of CpG oligonucleotide 7909 (PF-03512676) in patients with previously treated chronic lymphocytic leukemia. (2012) Leukemia & Lymphoma, 2012; 53(2): 211-217
29. Pawel Muranski & Nicholas P Restifo. Adoptive immunotherapy of cancer using CD4+ T cells. (2009) Current Opinion in Immunology, 21:200-208
30. Susan L. Swain, K. Kai McKinstry and Tara M. Strutt. Expanding roles for CD4+ T cells in immunity to viruses. (2012) Nature Reviews Immunology 12, pages 136-148
31. Richard Kennedyl, Esteban Celis, Multiple roles for CD4 T cells in anti-tumor immune responses. (2008) Immunological Reviews. 222, 129-144
32. Marij J. P. Welters, Gemma G. Kenter, Sytse J. Piersma, Annelies P. G. Vloon, Margriet J. G. Löwik, Dorien M. A. Berends-van der Meer, Jan W. Drijfhout, A. Rob P. M. Valentijn, Amon R. Wafelman, Jaap Oostendorp, Gert Jan Fleuren, Rienk Offringa, Cornelis J. M. Melief and Sjoerd H. van der Burg. Induction of Tumor-Specific CD4+ and CD8+ T-Cell Immunity in Cervical Cancer Patients by a Human Papillomavirus Type 16 E6 and E7 Long Peptides Vaccine. (2008) Clin Cancer Res. 14, 178-187.
33. Christopher A. Klebanoff Luca Gattinoni Nicholas P. Restifo. CD8+ T-cell memory in tumor immunology and immunotherapy. (2006) Immunological Reviews, 211, 214-224
34. Radovic-Moreno, A. F.; Chernyak, N.; Mader, C. C.; Nallagatla, S.; Kang, R. S.; Hao, L.; Walker, D. A.; Halo, T. L.; Merkel, T. J.; Rische, C. H.; et al. Immunomodulatory Spherical Nucleic Acids. Proc. Natl. Acad. Sci. U.S.A 2015, 112 (13), 3892-3897.

What is claimed is:

1. An immunostimulatory protein-core spherical nucleic acid (IP-SNA) comprising:
a protein core; and
a shell of oligonucleotides attached to the protein core, wherein
the shell of oligonucleotides comprises a ratio of immunostimulatory oligonucleotides to non-immunostimulatory oligonucleotides that is between 100:1 and 1:100.

2. The IP-SNA of claim 1, wherein the protein comprises an antigen that is a tumor associated antigen, a tumor specific antigen, a viral antigen, a neoantigen, or a combination thereof.

3. The IP-SNA of claim 2, wherein the antigen is OVA1, MSLN, p53, Ras, a melanoma related antigen, a HPV related antigen, a prostate cancer related antigen, an ovarian cancer related antigen, a breast cancer related antigen, a hepatocellular carcinoma related antigen, a bowel cancer related antigen, a lung cancer related antigen, an osteocarcinoma related antigen, human papillomavirus (HPV) E6/E7 nuclear protein, or a combination thereof.

4. The IP-SNA of any one of claims 1-3, wherein the ratio of immunostimulatory oligonucleotides to non-immunostimulatory oligonucleotides is about 2:5.

5. The IP-SNA of any one of claims 1-3, wherein the ratio of immunostimulatory oligonucleotides to non-immunostimulatory oligonucleotides is about 1:1.

6. The IP-SNA of any one of claims 1-3, wherein the ratio of immunostimulatory oligonucleotides to non-immunostimulatory oligonucleotides is about 2:4.

7. The IP-SNA of any one of claims 1-6, wherein at least one oligonucleotide of the shell of oligonucleotides is attached to the protein core through a linker.

8. The IP-SNA of claim 7, wherein the linker is a cleavable linker, a non-cleavable linker, a traceless linker, or a combination thereof.

9. The IP-SNA of claim 7 or claim 8, wherein the linker is a carbamate alkylene dithiolate linker.

10. The IP-SNA of claim 9, wherein at least one oligonucleotide of the shell of oligonucleotides comprises protein-core-NH—C(O)—O—C₂₋₅alkylene-S—S—C₂₋₇alkylene-Oligonucleotide, or protein-core-NH—C(O)—O—CH₂—Ar—S—S—C₂₋₇alkylene-Oligonucleotide, and Ar comprises a meta- or para-substituted phenyl.

11. The IP-SNA of claim 9, wherein at least one oligonucleotide of the shell of oligonucleotides comprises protein-core-NH—C(O)—O—C(ZA)(ZB)C₁₋₄alkylene-C(XA)(XB)—S—S—C(YA)(YB)C₁₋₆alkylene-Oligonucleotide, and ZA, ZB, XA, XB, YA, and YB are each independently H, Me, Et, or Pr.

12. The IP-SNA of claim 9, wherein at least one oligonucleotide of the shell of oligonucleotides comprises protein-core-NH—C(O)—O—C(XA)(XB)—Ar—S—S—C(YA)(YB)C₂₋₆alkylene-Oligonucleotide, and XA, XB, YA, and YB are each independently H, Me, Et, or iPr.

13. The IP-SNA of any one of claims 1-8, wherein the linker is an amide alkylene dithiolate linker.

14. The IP-SNA of claim 13, wherein at least one oligonucleotide of the shell of oligonucleotides comprises protein-core-NH—C(O)— C₂₋₅alkylene-S—S—C₂₋₇alkylene-Oligonucleotide.

15. The IP-SNA of claim 13, wherein at least one oligonucleotide of the shell of oligonucleotides comprises protein-core-NH—C(O)— C₁₋₄alkylene-C(XA)(XB)—S—S—C(YA)(YB)C₁₋₆alkylene-Oligonucleotide, and XA, XB, YA and YB are each independently H, Me, Et, or iPr.

16. The IP-SNA of any one of claims 1-8, wherein the linker is an amide alkylene thioether linker.

17. The IP-SNA of claim 16, wherein at least one oligonucleotide of the shell of oligonucleotides comprises protein-core-NH—C(O)— C₂₋₄alkylene-N-succinimidyl-S—C₂₋₆alkylene-Oligonucleotide.

18. The IP-SNA of any one of claims 1-17, wherein each of the immunostimulatory oligonucleotides is a toll-like receptor (TLR) agonist.

19. The IP-SNA of claim 18, wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

20. The IP-SNA of any one of claims 1-19, wherein each of the immunostimulatory oligonucleotides comprises a CpG nucleotide sequence.

21. The IP-SNA of any one of claims 1-20, wherein each of the non-immunostimulatory oligonucleotides comprises a sequence that is 5′-TTTTTTTTTTTTTTTTTTTT-Spacer 18-3′ (“T20”; SEQ ID NO: 1), 5′-(GGT)₇-hexaethyleneglycol-3′ (SEQ ID NO: 2), 5′-AAAAAAAAAAAAAAAAAAAA-hexaethyleneglycol-3′ (“A20”; SEQ ID NO: 3), or 5′-(AAT)₇-Spacer 18-3′ (SEQ ID NO: 4).

22. The IP-SNA of any one of claims 1-21, wherein at least one oligonucleotide in the shell of oligonucleotides is single-stranded DNA or double-stranded DNA.

23. The IP-SNA of any one of claims 1-21, wherein at least one oligonucleotide in the shell of oligonucleotides is single-stranded RNA or double-stranded RNA.

24. The IP-SNA of any one of claims 1-23, wherein the shell of oligonucleotides comprises about 2 to about 20 oligonucleotides.

25. The IP-SNA of any one of claims 1-24, wherein the shell of oligonucleotides comprises 7 oligonucleotides.

26. The IP-SNA of any one of claims 1-25, wherein the shell of oligonucleotides consists of 7 oligonucleotides.

27. A composition comprising a plurality of the immunostimulatory protein-core spherical nucleic acids (IP-SNAs) of any one of the preceding claims.

28. The composition of claim 27, wherein at least two of the IP-SNAs comprise a different protein core.

29. A pharmaceutical formulation comprising the immunostimulatory protein-core SNA (IP-SNA) of any one of claims 1-26 or the composition of claim 27 or claim 28, and a pharmaceutically acceptable carrier or diluent.

30. An antigenic composition comprising the immunostimulatory protein-core SNA (IP-SNA) of any one of claims 1-26 or the composition of claim 27 or claim 28 in a pharmaceutically acceptable carrier, diluent, stabilizer, preservative, or adjuvant, or the pharmaceutical formulation of claim 29, wherein the antigenic composition is capable of generating an immune response including antibody generation or a protective immune response in a mammalian subject.

31. The antigenic composition of claim 30, wherein the antibody response is a neutralizing antibody response or a protective antibody response.

32. A method of producing an immune response to a disease in a subject, comprising administering to the subject an effective amount of the antigenic composition of claim 30 or claim 31, thereby producing an immune response to the disease in the subject.

33. The method of claim 32, wherein the disease is an infection or an immunodeficiency disease.

34. The method of claim 32, wherein the disease is cancer.

35. The method of claim 34, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

36. A method of treating a disease in a subject in need thereof, comprising administering to the subject an effective amount of the immunostimulatory protein-core SNA (IP-SNA) of any one of claims 1-26, the composition of claim 27 or claim 28, the pharmaceutical formulation of claim 29, or the antigenic composition of claim 30 or claim 31, thereby treating the disease in the subject.

37. The method of claim 36, wherein the disease is an infection or an immunodeficiency disease.

38. The method of claim 37 wherein the infection is Anthrax, Chickenpox, Common cold, Diphtheria, E. coli infection, Giardiasis, HIV/AIDS, Infectious, mononucleosis, Influenza (flu), Lyme disease, Malaria, Measles, Meningitis, Mumps, Poliomyelitis (polio), Pneumonia, Rocky mountain spotted fever, Rubella (German measles), Salmonella infections, Severe acute respiratory syndrome (SARS), Sexually transmitted diseases, Shingles (herpes zoster), Tetanus, Toxic shock syndrome, Tuberculosis, Viral hepatitis, West Nile virus, Whooping cough (pertussis), or a combination thereof.

39. The method of claim 37 or claim 38, wherein the immunodeficiency disease is ataxia-telangiectasia, chediak-Higashi syndrome, combined immunodeficiency disease, complement deficiencies, DiGeorge syndrome, hypogammaglobulinemia, Job syndrome, leukocyte adhesion defects, panhypogamma globulinemia, Bruton's disease, congenital agammaglobulinemia, selective deficiency of IgA, Wiskott-Aldrich syndrome, or a combination thereof.

40. The method of any one of claims 36-39, wherein the disease is cancer.

41. The method of claim 40, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

42. The method of any one of claims 36-39, wherein the administering is subcutaneous, intravenous, intraperitoneal, intranasal, or intramuscular.

43. A method of stimulating a CD8 T-Cell response in a subject having cancer, comprising administering to the subject an effective amount of the immunostimulatory protein-core SNA (IP-SNA) of any one of claims 1-26, the composition of claim 27 or claim 28, the pharmaceutical formulation of claim 29, or the antigenic composition of claim 30 or claim 31, wherein the ratio of immunostimulatory oligonucleotides to non-immunostimulatory oligonucleotides is less than or equal to 1, thereby stimulating the CD8 T-cell response in the subject.

44. The method of claim 43, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

45. The method of claim 43 or claim 44, wherein the ratio of immunostimulatory oligonucleotides to non-immunostimulatory oligonucleotides is about 1:1.

46. The method of claim 43 or claim 44, wherein the ratio of immunostimulatory oligonucleotides to non-immunostimulatory oligonucleotides is about 2:5.

47. A method of stimulating a CD4 T-Cell response in a subject having a viral infection, comprising administering to the subject an effective amount of the immunostimulatory protein-core SNA (IP-SNA) of any one of claims 1-26, the composition of claim 27 or claim 28, the pharmaceutical formulation of claim 29, or the antigenic composition of claim 30 or claim 31, wherein the ratio of immunostimulatory oligonucleotides to non-immunostimulatory oligonucleotides is greater than 1, thereby stimulating the CD4 T-cell response in the subject.

48. The method of claim 47, wherein the ratio of immunostimulatory oligonucleotides to non-immunostimulatory oligonucleotides is about 1:0.

49. The method of claim 47, wherein the ratio of immunostimulatory oligonucleotides to non-immunostimulatory oligonucleotides is about 2:1.

50. The method of any one of claims 47-49, wherein the viral infection is influenza, HIV, pneumonia virus, human papilloma virus (HPV), or a virus that causes cancer.

51. The method of claim 50, wherein the virus that causes cancer is human papilloma virus, Epstein-Barr virus, hepatitis B virus, human herpes virus-8, hepatitis C virus, or a combination thereof.
